(19) United States
(12) Patent Application Publication
(10) Pub. No.: US 2026/0038689 A1
(43) Pub. Date: Feb. 5, 2026

(54) SYSTEMS AND METHODS FOR PREDICTION OF MEDICAL DISEASES

(71) Applicant: nference, Inc., Cambridge, MA (US)

(72) Inventors: Rakesh Barve, Bengaluru (IN); Tyler Wagner, Boston, MA (US); Samir Awasthi, Boston, MA (US); Praveen Kumar M, Cambridge, MA (US); Sairam Bade, Bengaluru (IN)

(73) Assignee: nference, Inc., Cambridge, MA (US)

(21) Appl. No.: 18/794,664

(22) Filed: Aug. 5, 2024

(51) Int. Cl. G16H 50/30; G16H 50/70

(52) U.S. Cl. CPC G16H 50/30; G16H 50/70

(57) ABSTRACT

A system for prediction of medical diseases including a computing device configured to receive electronic health records, identify a presence of a medical diagnosis, wherein determining the diagnosis includes identifying medical factors within each electronic health record and assigning the medical diagnosis to each electronic health record, generate medical training data as a function of the electronic health records and the presence of the medical diagnosis, wherein the medical training data includes electronic health records correlated to medical diagnoses, and wherein at least a portion of the electronic health records lack a medical diagnosis and train one or more medical machine learning models as a function of the medical training data, wherein the one or more medical machine learning models are configured to receive an electronic health record associated with a patient as an input and output a probability of medical determination.

### FIELD OF THE INVENTION

The present invention generally relates to the field of machine learning models. In particular, the present invention is directed to prediction of medical diseases.

### BACKGROUND

Treatment for medical diseases can be effective if the disease can be properly detected in advance. However, current systems used to detect medical diseases earlier are ineffective. By way of example, Barrett's esophagus (BE) is a precursor to esophageal adenocarcinoma (EAC), a lethal form of cancer. Screening for BE is recommended in individuals with multiple risk factors, but current risk prediction tools have limited accuracy and are not widely implemented in clinical practice. This is due to the complexity of integrating multiple risk factors and the time-consuming nature of the assessment process. Additionally, the invasive and expensive nature of endoscopy, which is used for screening, further hinders its utilization.

### SUMMARY OF THE DISCLOSURE

In an aspect, a system for prediction of medical diseases is described. The system includes at least a processor and a memory communicatively connected to the at least a processor. The memory contains instructions configuring the at least a processor to receive a plurality of electronic health records associated with a plurality of patients from a patient database, identify a presence of a medical diagnosis for each electronic health record of the plurality of electronic health records, wherein determining the medical diagnosis includes identifying one or more medical factors within each heath record and assigning the medical diagnosis to each electronic health record as a function of the one or more medical factors. The processor is further configured to generate medical training data as a function of the electronic health records and the presence of the medical diagnosis including a plurality of electronic health records correlated to a plurality of medical diagnoses wherein at least a portion of the plurality of electronic health records lack a medical diagnosis and train one or more medical machine learning models as a function of the medical training data, wherein the one or more medical machine learning models are configured to receive an electronic health record associated with a patient as an input and output a probability of medical determination.

In another aspect, a method for prediction of medical diseases is described, The method includes receiving, by at least a processor, a plurality of electronic health records associated with a plurality of patients from a patient database and identifying, by the at least a processor, a presence of a medical diagnosis for each electronic health record of the plurality of electronic health records, wherein determining the medical diagnosis includes identifying one or more medical factors within each electronic heath record and assigning the medical diagnosis to each electronic health record as a function of the one or more medical factors. The method further includes generating, by the at least a processor, medical training data as a function of the electronic health records and the presence of the medical diagnosis wherein the gastro intestinal training data includes a plurality of electronic health records correlated to a plurality of medical diagnoses wherein at least a portion of the plurality of electronic health records lack a medical diagnosis and training, by the at least a processor, one or more medical machine learning models as a function of the medical training data, wherein the one or more medical machine learning models are configured to receive an electronic health record associated with a patient as an input and output a probability of medical determination.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is an exemplary embodiment of a system for prediction of medical diseases;

FIG. 2 shows an illustrative embodiment of a method for identification of disease cohorts;

FIG. 3 shows an illustrative embodiment of a process of data extraction from the observation time of a hypothetical patient;

FIG. 4 shows an illustrative embodiment of a method for processing a sequence of events in a patient timeline;

FIG. 5 shows an illustrative embodiment of a schematic illustrating the data sets used for a model training process;

FIG. 6 shows a set of sequential steps taken for inclusion and exclusion criteria and corresponding case counts in the form of a table;

FIG. 7 shows baseline characteristics of the case and control cohorts identified using the electronic search strategy are presented in the form of a table;

FIG. 8 shows performance characteristics of BE and EAC prediction models presented in the form of a table;

FIG. 9 is a block diagram of an exemplary machine-learning process;

FIG. 10 is a diagram of an exemplary embodiment of a neural network;

FIG. 11 is a diagram of an exemplary embodiment of a node of a neural network;

FIGS. 12A-C are exemplary embodiments of a schematic of data management, quality control and validation process;

FIG. 13 is an exemplary embodiment of a categorization of lab tests based on a standard normal distribution across all Mayo patients;

FIG. 14 is an exemplary embodiment of a schematic of PH-COPD, COPD-only and screening COPD cohorts;

FIG. 15 is an exemplary embodiment of a result for multivariate logistic regression predicting PH-COPD versus COPD only;

FIG. 16 is a flow diagram illustrating an exemplary embodiment of a method for prediction of medical diseases; and

FIG. 17 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

There is a need for a more accurate and efficient risk prediction models that can be easily integrated into electronic health records (EHR) and facilitate the implementation of minimally invasive screening technologies. Machine learning models developed using EHR data have the potential to address these challenges by automatically incorporating multiple risk factors and improving the accuracy of BE/EAC risk prediction. At a high level, aspects of the present disclosure are directed to systems and methods for prediction of gastrointestinal diseases such as BE and EAC. Systems and methods described herein include process of identifying patients who were diagnosed with BE and EAC. This involved a mix of natural language processing (NLP) algorithms running on patient notes and structured EHR data, as described below. Patients diagnosed with BE and EAC from CDAP formed the 2 disease-positive cohorts. A randomly selected set of patients who did not have BE/EAC and who were propensity matched with the disease-positive cohorts (see gastrointestinal cohort below) formed the control cohort. Two predictive models, 1 for each disease (BE and EAC), were developed using these cohorts. The models were built to predict the probability that a patient would develop the disease at least 1 year before diagnosis. This may be achieved by including only patient data between 1 and 5 years before the diagnosis of BE or EAC (the observation period) for model development, allowing for minimization of protopathic bias. Aspects of this disclosure can be used to make predictions as to the probability that a patient may have BE and/or AEC in the coming future. Exemplary embodiments illustrating aspects of the present disclosure are described below in the context of several specific examples.

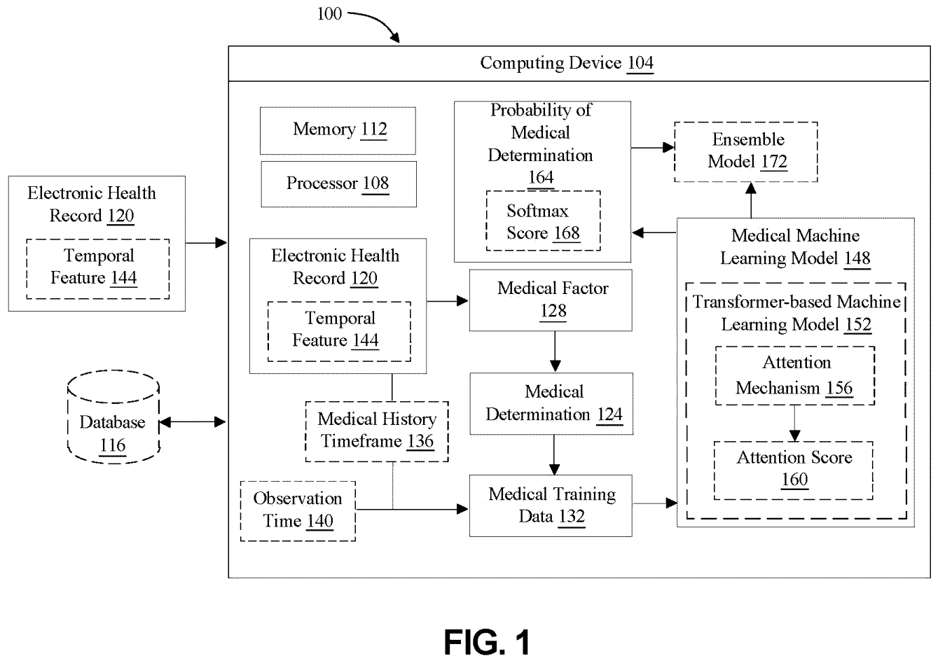

Referring now to FIG. 1, a system 100 for prediction of medical conditions is described. In one or more embodiments, system 100 may be configured to predict any medical condition and/or medical disease. System 100 includes a computing device 104. System 100 includes a processor 108. Processor 108 may include, without limitation, any processor 108 described in this disclosure. Processor 108 may be included in a and/or consistent with computing device 104. In one or more embodiments, processor 108 may include a multi-core processor. In one or more embodiments, multi-core processor may include multiple processor cores and/or individual processing units. “Processing unit” for the purposes of this disclosure is a device that is capable of executing instructions and performing calculations for a computing device 104. In one or more embodiments, processing units may retrieve instructions from a memory, decode the data, secure functions and transmit the functions back to the memory. In one or more embodiments, processing units may include an arithmetic logic unit (ALU) wherein the ALU is responsible for carrying out arithmetic and logical operations. This may include, addition, subtraction, multiplication, comparing two data, contrasting two data and the like. In one or more embodiments, processing unit may include a control unit wherein the control unit manages execution of instructions such that they are performed in the correct order. In none or more embodiments, processing unit may include registers wherein the registers may be used for temporary storage of data such as inputs fed into the processor and/or outputs executed by the processor. In one or more embodiments, processing unit may include cache memory wherein memory may be retrieved from cache memory for retrieval of data. In one or more embodiments, processing unit may include a clock register wherein the clock register may be configured to synchronize the processor with other computing components. In one or more embodiments, processor 108 may include more than one processing unit having at least one or more arithmetic and logic units (ALUs) with hardware components that may perform arithmetic and logic operations. Processing units may further include registers to hold operands and results, as well as potentially “reservation station” queues of registers, registers to store interim results in multi-cycle operations, and an instruction unit/control circuit (including e.g. a finite state machine and/or multiplexor) that reads op codes from program instruction register banks and/or receives those op codes and enables registers/arithmetic and logic operators to read/output values. In one or more embodiments, processing unit may include a floating-point unit (FPU) wherein the FPU may be configured to handle arithmetic operations with floating point numbers. In one or more embodiments, processor 108 may include a plurality of processing units wherein each processing unit may be configured for a particular task and/or function. In one or more embodiments, each core within multi-core processor may function independently. In one or more embodiments, each core within multi-core processor may perform functions in parallel with other cores. In one or more embodiments, multi-core processor may allow for a dedicated core for each program and/or software running on a computing system. In one or more embodiments, multiple cores may be used for a singular function and/or multiple functions. In one or more embodiments, multi-core processor may allow for a computing system to perform differing functions in parallel. In one or more embodiments, processor 108 may include a plurality of multi-core processors. Computing device 104 may include any computing device as described in this disclosure, including without limitation a microcontroller, microprocessor, digital signal processor (DSP) and/or system on a chip (SoC) as described in this disclosure. Computing device 104 may include, be included in, and/or communicate with a mobile device such as a mobile telephone or smartphone. Computing device 104 may include a single computing device 104 operating independently or may include two or more computing devices operating in concert, in parallel, sequentially or the like; two or more computing devices may be included together in a single computing device 104 or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device 104. Computing device 104 may include but is not limited to, for example, a computing device 104 or cluster of computing devices in a first location and a second computing device 104 or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device 104, which may operate in parallel, in series, redundantly, or in any other manner used for distribution of tasks or memory 112 between computing devices. Computing device 104 may be implemented, as a non-limiting example, using a “shared nothing” architecture.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, computing device 104 may perform determinations, classification, and/or analysis steps, methods, processes, or the like as described in this disclosure using machine-learning processes. A “machine-learning process,” as used in this disclosure, is a process that automatedly uses a body of data known as “training data” and/or a “training set” (described further below in this disclosure) to generate an algorithm that will be performed by a Processor module to produce outputs given data provided as inputs; this is in contrast to a non-machine learning software program where the commands to be executed are determined in advance by a user and written in a programming language. A machine-learning process may utilize supervised, unsupervised, lazy-learning processes and/or neural networks, described further below.

With continued reference to FIG. 1, system 100 includes a memory 112 communicatively connected to processor 108, wherein the memory 112 contains instructions configuring processor 108 to perform any processing steps as described herein. As used in this disclosure, “communicatively connected” means connected by way of a connection, attachment, or linkage between two or more relata which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, using a bus or other facility for intercommunication between elements of a computing device 104. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

With continued reference to FIG. 1, memory 112 may include a primary memory and a secondary memory. “Primary memory” also known as “random access memory” (RAM) for the purposes of this disclosure is a short-term storage device in which information is processed. In one or more embodiments, during use of computing device 104, instructions and/or information may be transmitted to primary memory wherein information may be processed. In one or more embodiments, information may only be populated within primary memory while a particular software is running. In one or more embodiments, information within primary memory is wiped and/or removed after computing device 104 has been turned off and/or use of a software has been terminated. In one or more embodiments, primary memory may be referred to as “Volatile memory” wherein the volatile memory only holds information while data is being used and/or processed. In one or more embodiments, volatile memory may lose information after a loss of power. “Secondary memory” also known as “storage,” “hard disk drive” and the like for the purposes of this disclosure is a long-term storage device in which an operating system and other information is stored. In one or remote embodiments, information may be retrieved from secondary memory and transmitted to primary memory during use. In one or more embodiments, secondary memory may be referred to as non-volatile memory wherein information is preserved even during a loss of power. In one or more embodiments, data within secondary memory cannot be accessed by processor. In one or more embodiments, data is transferred from secondary to primary memory wherein processor 108 may access the information from primary memory.

Still referring to FIG. 1, system 100 may include a database 116. Database may include a remote database 116. Database 116 may be implemented, without limitation, as a relational database, a key-value retrieval database such as a NOSQL database, or any other format or structure for use as database that a person skilled in the art would recognize as suitable upon review of the entirety of this disclosure. Database may alternatively or additionally be implemented using a distributed data storage protocol and/or data structure, such as a distributed hash table or the like. Database 116 may include a plurality of data entries and/or records as described above. Data entries in database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in database may store, retrieve, organize, and/or reflect data and/or records.

With continued reference to FIG. 1, system 100 may include and/or be communicatively connected to a server, such as but not limited to, a remote server, a cloud server, a network server and the like. In one or more embodiments. In one or more embodiments, computing device 104 may be configured to transmit one or more processes to be executed by server. In one or more embodiments, server may contain additional and/or increased processor power wherein one or more processes as described below may be performed by server. For example, and without limitation, one or more processes associated with machine learning may be performed by network server, wherein data is transmitted to server, processed and transmitted back to computing device. In one or more embodiments, server may be configured to perform one or more processes as described below to allow for increased computational power and/or decreased power usage by system computing device 104. In one or more embodiments, computing device 104 may transmit processes to server wherein computing device 104 may conserve power or energy.

With continued reference to FIG. 1, processor 108 is configured to receive a plurality of electronic health records 120 associated with a plurality of patients. An “electronic health record,” for the purposes of this disclosure, is digital information associated with an individual's health. For example, and without limitation, electronic health record 120 may include medications an individual is taking, recent diagnosis, recent treatment, laboratory test results and the like. In one or more embodiments, an individual may seek medical treatment wherein information about the patient may be recorded in electronic health record 120 during and/or following treatment. A “patient” for the purposes of this disclosure is any individual currently seeking or has previously sought medical treatment. For example, and without limitation, patient may include an individual who has sought treatment in the past, who has undergone medical testing and the like. In one or more embodiments, a patient may include an individual who has previously sought medical treatment. In one or more embodiments, electronic health record 120 may include patient demographics. In one or more embodiments, patient demographics may include information about the patient's age, sex, race/ethnicity, family history of Barrett's esophagus (BE) or esophageal adenocarcinoma (EAC) and the like. In one or more embodiments, electronic health record 120 may include the medical history of a patient. Medical history may include but is not limited to information about the patient's medical history, such as comorbidities (e.g., coronary artery disease), symptoms (e.g., heartburn, dyspepsia), previous diagnoses (e.g., gastroesophageal reflux disease), previous medical treatments, previous weight, previous height and the like. In one or more embodiments, electronic health record 120 may include medications taken by the patient and/or prescribed to the patient. In one or more embodiments, medications may include treatment plans, dosing of medication and the like. In one or more embodiments, electronic health records 120 may include laboratory tests. Laboratory rests may include results such as blood tests (e.g., hemoglobin, cholesterol levels) and electrolyte levels. In one or more embodiments, laboratory tests may include any information typically given as part of a blood test, urine test and the like. In one or more embodiments, electronic health record 120 may include medical procedures. Medical procedures may provide information about any endoscopy procedures the patient has undergone, as well as the presence of specific keywords in pathology notes. In one or more embodiments, electronic health record 120 may further include risk factors as determined by a physician and indicated within electronic health record 120. Rick factors may include established risk factors for BE and EAC, such as age, sex, smoking status, and obesity. In one or more embodiments, electronic health record 120 may include any information associated with an individual seeking or who has sought medical treatment. In one or more embodiments, electronic health records 120 may include any medical information associated with a patient that is received in the ordinary course of medical treatment.

With continued reference to FIG. 1, each electronic health record 120 may be associated with a single patient wherein a plurality of health records may be associated with a plurality of patients. In one or more embodiments, electronic health record 120 may include prescriptions written, notes written by a physician, scans of medical documents and the like. In one or more embodiments, each electronic health record 120 may include dates associated with each element within electronic health record 120. For example, and without limitation, electronic health record 120 may include a date in which a mediation was prescribed, a date in which laboratory testing was conducted, a date in which treatment was provided, a date in which a diagnosis was given and the like. In one or more embodiments, any information recorded within electronic health record 120 may include a date of recordation. In one or more embodiments, laboratory results, medication, treatment, doctors, visits, recordation of previous medical history and the like may have a date indicating when the information was recorded.

With continued reference to FIG. 1, electronic health records 120 may be received from a database such as patient database 116. A “patient database,” for the purposes of this disclosure, is a database having medical information associated with patients. In one or more embodiments, patient database may include an EHR database and/or any other database that is configured to store electronic health records. In one or more embodiments, database 116 may include patient database 116. In one or more embodiments, patient database 116 may include a plurality of electronic health records 120 of a plurality of patients. In one or more embodiments, patient database 116 may be iteratively updated with new information associated with patients, such as for example, new medications prescribed, new treatments provided and the like. In one or more embodiments, electronic health record 120 may be received from an EHR database 116. In an embodiment, an HER database 116 may include a collection of patient databases 116 that contain information associated with a patients health. In one or more embodiments, individuals may be given access to EHR database 116 such that individuals may access electronic health records 120. In one or more embodiments, EHR database 116 may include de-identified information wherein de-identified information includes information in which identifiers have been removed. For example, and without limitation, de-identified information may include an electronic health record 120 in which the name of the patient, social security number of the patient and/or any other information that may be used to identify the patients, is removed. In one or more embodiments, electronic heath records may be de-identified wherein information used to identify the patient is removed. In one or more embodiments, plurality of electronic health records 120 may be received from a user of system, a medical professional and the like.

With continued reference to FIG. 1, processor 108 is configured to identify and/or determine a presence or likelihood of presence of a medical condition for each electronic health record 120. A “medical determination,” for the purposes of this disclosure, is a determination of whether a patient is suffering or has suffered from any known medical condition. For example, and without limitation, medical diagnosis may include an indication that a patient is suffering or has suffered from diabetes, a stroke, cancer, hypertension, coronary artery disease, migraine, Alzheimer's disease, hyperthyroidism, ovarian cancer, prostate cancer, gastric ulcer, Crohn's disease celiac disease, Parkinson's disease, chronic kidney disease, and the like. In one or more embodiments, medical determination 124 may include a gastrointestinal determination. In one or more embodiments, gastrointestinal determination and medical determination may be used interchangeably throughout this disclosure. In one or more embodiments, steps, process and the like used in reference to gastrointestinal determination may be used for any medical determination. In one or more embodiments, processor 108 is configured to determine and/or identify a presence of medical determination 124, e.g., gastrointestinal determination, for each electronic health record 120. A “gastrointestinal determination” for the purposes of this disclosure is a determination of whether a patient suffers from a particular gastrointestinal disease or a probability that the patient is developing a gastrointestinal disease. For example, and without limitation, gastrointestinal determination may include a determination that a patient suffers, or potentially suffers, from Barrett's esophagus (BE). In some embodiments, medical determination 124 may include medical determinations related to cardiac diseases, hyperkalemia, and the like. In one or more embodiments, gastrointestinal diseases may include but are not limited to, Gastroesophageal reflux disease (GERD), peptic ulcer disease, esophageal cancer, hiatal Hernia, esophageal adenocarcinoma (EAC) and the like. In one or more embodiments, gastrointestinal determination may include any determination or indication that a patient suffers from a gastrointestinal disease. In one or more embodiments, processor 108 may be configured to determine medical determination 124 by labeling each health record with a medical disease, if any. For example, and without limitation, an electronic health record 120 may include a label indicating ‘Barrett's esophagus’ wherein the label may indicate that the patient has been diagnosed with and/or suffers from Barrett's esophagus. Additionally or alternatively, an electronic health record 120 may lack a label and/or contain a label indicating that no gastrointestinal diseases or no medical diseases were found. In one or more embodiments, following the determination, a plurality of electronic health records 120 may exist either containing a label indicating the gastrointestinal disease or containing a label indicating that the patient does not suffer from a gastrointestinal disease.

With continued reference to FIG. 1, processor 108 may use diagnosis codes listed within electronic health records 120 to determine gastrointestinal determination and/or medical determination 124. The International Classification of Diseases (ICD-9 and ICD-10), Systematized Nomenclature of Medicine, and Hospital Adaptation of the International Classification of Diseases codes may be used to identify individuals within gastrointestinal diseases. In one or more embodiments, diagnosis codes may include unique identifiers to classify disease, injuries, symptoms, and other health conditions. In one or more embodiments, diagnosis codes may be used to standardize medical records such that physicians would not be required to write out a diagnosis in full detail. In one or more embodiments, electronic health records 120 may include a plurality of diagnosis codes wherein processor 108 may identify diagnosis codes associated with gastrointestinal diseases. In one or more embodiments, processor 108 may be configured to query each electronic health record 120 to located diagnosis codes associated with a gastrointestinal disease. In one or more embodiments, processor 108 may be configured to search electronic health records 120 for a plurality of diagnoses and/or diseases aside from gastrointestinal diseases. In one or more embodiments, processor 108 may be configured to remove electronic health records 120 from plurality of health records in instances in which a diagnosis code indicates a differing disease. In one or more embodiments, only patients having a gastrointestinal disease may be of importance wherein other patients diagnosed with differing conditions may be removed from plurality of health records. In one or more embodiments, processor 108 may be configured to retain electronic health records 120 having diagnosis codes associated with gastrointestinal disease while discarding other electronic health records 120. In one or more embodiments, processor 108 may be configured to label electronic health records 120 having the correct diagnosis code such that electronic health records 120 may be distinguished. In one or more embodiments, processor 108 may further be configured to identify patients who have not been diagnosed with gastrointestinal disease by confirming that such electronic health records 120 lack the proper diagnosis code.

With continued reference to FIG. 1, processor 108 may determine medical determination 124 through the identification of procedure codes. In one or more embodiments procedure codes may include unique identifiers used to indicate that a particular procedure had occurred or will occur. In one or more embodiments, processor 108 may be configured to identify procedure codes that may have been performed due to diagnosis of a gastrointestinal disease. For example, and without limitation, processor 108 may identify an endoscopy procedure code, preceding the diagnosis of BE or EAC. In one or more embodiments, the presence of a particular procedure within electronic health record 120 may indicate that the patient was diagnosed with a gastrointestinal disease. In one or more embodiment, processor 108 may be configured to identify an endoscopy procedure code, preceding a diagnosis of BE or EAC.

With continued reference to FIG. 1, processor 108 is configured to identify one or more medical factors 128 within each health record. In one or more embodiments, processor 108 is configured to identify medical factors within each health record. “Medical factors” for the purposes of this disclosure refers to any information that is indicative that a patient suffers from a particular medical disease. For example, and without limitation, low insulin levels may include a medical factor for a medical disease of diabetes. In one or more embodiments, medical factors 128 may include gastrointestinal factors. In one or more embodiments, gastrointestinal factors may be used throughout this disclosure by way of example. In one or more embodiments, steps and/or process used throughout this disclosure for gastrointestinal factors may be used for identification of any medical factors. A “gastrointestinal factor,” for the purposes of this disclosure, is information that is indicative that a patient suffers from a gastrointestinal disease. For example and without limitation, medical factor 128 may include disease codes associated with gastrointestinal diseases or other diseases as described above. In one or more embodiments, medical factors 128 may further include medication codes, treatment codes, procedures codes, diseases codes and the like associated with a medical disease. In an embodiment, an electronic health record 120 having a disease code, procedure code and the like associated with a medical disease may indicate that the patient suffers from the medical disease. In one or more embodiments, medical factors 128 may include synonyms associated with medical diseases in order to determine whether a patient suffers from a medical disease. For example, and without limitation, the phrases “adenocarcinoma of the esophagus” and/or “esophageal adenocarcinoma” may be used to indicate that the patient suffers from BE and/or EAC. In one or more embodiments, processor 108 may be configured to identify words and/or phrases most commonly used with each medical disease. In one or more embodiments, processor 108 may be configured to identify words or phrases describing symptoms most closely related to a medical disease. For example and without limitation a patient describing “burning in the throat” may indicate that the patient suffers from acid reflux or GERD. In one or more embodiments, processor 108 may use a large language model to each for words, phrases, synonyms and the like and classify them to medical diseases.

Still referring to FIG. 1, system 100 may include and/or be communicatively connected to a large language model (LLM). A “large language model,” as used herein, is a deep learning data structure that can recognize, summarize, translate, predict and/or generate text and other content based on knowledge gained from massive datasets. Large language models may be trained on large sets of data. Training sets may be drawn from diverse sets of data such as, as non-limiting examples, novels, blog posts, articles, emails, unstructured data, electronic records, and the like. In some embodiments, training sets may include a variety of subject matters, such as, as nonlimiting examples, medical report documents, electronic health records 120, entity documents, business documents, inventory documentation, emails, user communications, advertising documents, newspaper articles, and the like. In some embodiments, training sets of an LLM may include information from one or more public or private databases 116. As a non-limiting example, training sets may include databases 116 associated with an entity. In some embodiments, training sets may include portions of documents associated with the electronic records 112 correlated to examples of outputs. In an embodiment, an LLM may include one or more architectures based on capability requirements of an LLM. Exemplary architectures may include, without limitation, GPT (Generative Pretrained Transformer), BERT (Bidirectional Encoder Representations from Transformers), T5 (Text-To-Text Transfer Transformer), and the like. Architecture choice may depend on a needed capability such generative, contextual, or other specific capabilities.

With continued reference to FIG. 1, in some embodiments, an LLM may be generally trained. As used in this disclosure, a “generally trained” LLM is an LLM that is trained on a general training set comprising a variety of subject matters, data sets, and fields. In some embodiments, an LLM may be initially generally trained. Additionally, or alternatively, an LLM may be specifically trained. As used in this disclosure, a “specifically trained” LLM is an LLM that is trained on a specific training set, wherein the specific training set includes data including specific correlations for the LLM to learn. As a non-limiting example, an LLM may be generally trained on a general training set, then specifically trained on a specific training set. In an embodiment, specific training of an LLM may be performed using a supervised machine learning process. In some embodiments, generally training an LLM may be performed using an unsupervised machine learning process. As a non-limiting example, specific training set may include information from a database 116. As a non-limiting example, specific training set may include text related to the users such as user specific data for electronic records correlated to examples of outputs. In an embodiment, training one or more machine learning models may include setting the parameters of the one or more models (weights and biases) either randomly or using a pretrained model. Generally training one or more machine learning models on a large corpus of text data can provide a starting point for fine-tuning on a specific task. A model such as an LLM may learn by adjusting its parameters during the training process to minimize a defined loss function, which measures the difference between predicted outputs and ground truth. Once a model has been generally trained, the model may then be specifically trained to fine-tune the pretrained model on task-specific data to adapt it to the target task. Fine-tuning may involve training a model with task-specific training data, adjusting the model's weights to optimize performance for the particular task. In some cases, this may include optimizing the model's performance by fine-tuning hyperparameters such as learning rate, batch size, and regularization. Hyperparameter tuning may help in achieving the best performance and convergence during training. In an embodiment, fine-tuning a pretrained model such as an LLM may include fine-tuning the pretrained model using Low-Rank Adaptation (LoRA). As used in this disclosure, “Low-Rank Adaptation” is a training technique for large language models that modifies a subset of parameters in the model. Low-Rank Adaptation may be configured to make the training process more computationally efficient by avoiding a need to train an entire model from scratch. In an exemplary embodiment, a subset of parameters that are updated may include parameters that are associated with a specific task or domain.

With continued reference to FIG. 1, in some embodiments an LLM may include and/or be produced using Generative Pretrained Transformer (GPT), GPT-2, GPT-3, GPT-4, and the like. GPT, GPT-2, GPT-3, GPT-3.5, and GPT-4 are products of Open AI Inc., of San Francisco, CA. An LLM may include a text prediction based algorithm configured to receive an article and apply a probability distribution to the words already typed in a sentence to work out the most likely word to come next in augmented articles. For example, if some words that have already been typed are “Nice to meet”, then it may be highly likely that the word “you” will come next. An LLM may output such predictions by ranking words by likelihood or a prompt parameter. For the example given above, an LLM may score “you” as the most likely, “your” as the next most likely, “his” or “her” next, and the like. An LLM may include an encoder component and a decoder component.

Still referring to FIG. 1, an LLM may include a transformer architecture. In some embodiments, encoder component of an LLM may include transformer architecture. A “transformer architecture,” for the purposes of this disclosure is a neural network architecture that uses self-attention and positional encoding. Transformer architecture may be designed to process sequential input data, such as natural language, with applications towards tasks such as translation and text summarization. Transformer architecture may process the entire input all at once. “Positional encoding,” for the purposes of this disclosure, refers to a data processing technique that encodes the location or position of an entity in a sequence. In some embodiments, each position in the sequence may be assigned a unique representation. In some embodiments, positional encoding may include mapping each position in the sequence to a position vector. In some embodiments, trigonometric functions, such as sine and cosine, may be used to determine the values in the position vector. In some embodiments, position vectors for a plurality of positions in a sequence may be assembled into a position matrix, wherein each row of position matrix may represent a position in the sequence.

With continued reference to FIG. 1, an LLM and/or transformer architecture may include an attention mechanism. An “attention mechanism,” as used herein, is a part of a neural architecture that enables a system to dynamically quantify the relevant features of the input data. In the case of natural language processing, input data may be a sequence of textual elements. It may be applied directly to the raw input or to its higher-level representation.

With continued reference to FIG. 1, attention mechanism may represent an improvement over a limitation of an encoder-decoder model. An encoder-decider model encodes an input sequence to one fixed length vector from which the output is decoded at each time step. This issue may be seen as a problem when decoding long sequences because it may make it difficult for the neural network to cope with long sentences, such as those that are longer than the sentences in the training corpus. Applying an attention mechanism, an LLM may predict the next word by searching for a set of positions in a source sentence where the most relevant information is concentrated. An LLM may then predict the next word based on context vectors associated with these source positions and all the previously generated target words, such as textual data of a dictionary correlated to a prompt in a training data set. A “context vector,” as used herein, are fixed-length vector representations useful for document retrieval and word sense disambiguation.

Still referring to FIG. 1, attention mechanism may include, without limitation, generalized attention self-attention, multi-head attention, additive attention, global attention, and the like. In generalized attention, when a sequence of words or an image is fed to an LLM, it may verify each element of the input sequence and compare it against the output sequence. Each iteration may involve the mechanism's encoder capturing the input sequence and comparing it with each element of the decoder's sequence. From the comparison scores, the mechanism may then select the words or parts of the image that it needs to pay attention to. In self-attention, an LLM may pick up particular parts at different positions in the input sequence and over time compute an initial composition of the output sequence. In multi-head attention, an LLM may include a transformer model of an attention mechanism. Attention mechanisms, as described above, may provide context for any position in the input sequence. For example, if the input data is a natural language sentence, the transformer does not have to process one word at a time. In multi-head attention, computations by an LLM may be repeated over several iterations, each computation may form parallel layers known as attention heads. Each separate head may independently pass the input sequence and corresponding output sequence element through a separate head. A final attention score may be produced by combining attention scores at each head so that every nuance of the input sequence is taken into consideration. In additive attention (Bahdanau attention mechanism), an LLM may make use of attention alignment scores based on a number of factors. Alignment scores may be calculated at different points in a neural network, and/or at different stages represented by discrete neural networks. Source or input sequence words are correlated with target or output sequence words but not to an exact degree. This correlation may take into account all hidden states and the final alignment score is the summation of the matrix of alignment scores. In global attention (Luong mechanism), in situations where neural machine translations are required, an LLM may either attend to all source words or predict the target sentence, thereby attending to a smaller subset of words.

With continued reference to FIG. 1, multi-headed attention in encoder may apply a specific attention mechanism called self-attention. Self-attention allows models such as an LLM or components thereof to associate each word in the input, to other words. As a non-limiting example, an LLM may learn to associate the word “you”, with “how” and “are”. It's also possible that an LLM learns that words structured in this pattern are typically a question and to respond appropriately. In some embodiments, to achieve self-attention, input may be fed into three distinct fully connected neural network layers to create query, key, and value vectors. Query, key, and value vectors may be fed through a linear layer; then, the query and key vectors may be multiplied using dot product matrix multiplication in order to produce a score matrix. The score matrix may determine the amount of focus for a word should be put on other words (thus, each word may be a score that corresponds to other words in the time-step). The values in score matrix may be scaled down. As a non-limiting example, score matrix may be divided by the square root of the dimension of the query and key vectors. In some embodiments, the softmax of the scaled scores in score matrix may be taken. The output of this softmax function may be called the attention weights. Attention weights may be multiplied by your value vector to obtain an output vector. The output vector may then be fed through a final linear layer.

Still referencing FIG. 1, in order to use self-attention in a multi-headed attention computation, query, key, and value may be split into N vectors before applying self-attention. Each self-attention process may be called a “head.” Each head may produce an output vector and each output vector from each head may be concatenated into a single vector. This single vector may then be fed through the final linear layer discussed above. In theory, each head can learn something different from the input, therefore giving the encoder model more representation power.

With continued reference to FIG. 1, encoder of transformer may include a residual connection. Residual connection may include adding the output from multi-headed attention to the positional input embedding. In some embodiments, the output from residual connection may go through a layer normalization. In some embodiments, the normalized residual output may be projected through a pointwise feed-forward network for further processing. The pointwise feed-forward network may include a couple of linear layers with a ReLU activation in between. The output may then be added to the input of the pointwise feed-forward network and further normalized.

Continuing to refer to FIG. 1, transformer architecture may include a decoder. Decoder may a multi-headed attention layer, a pointwise feed-forward layer, one or more residual connections, and layer normalization (particularly after each sub-layer), as discussed in more detail above. In some embodiments, decoder may include two multi-headed attention layers. In some embodiments, decoder may be autoregressive. For the purposes of this disclosure, “autoregressive” means that the decoder takes in a list of previous outputs as inputs along with encoder outputs containing attention information from the input.

With further reference to FIG. 1, in some embodiments, input to decoder may go through an embedding layer and positional encoding layer in order to obtain positional embeddings. Decoder may include a first multi-headed attention layer, wherein the first multi-headed attention layer may receive positional embeddings.

With continued reference to FIG. 1, first multi-headed attention layer may be configured to not condition to future tokens. As a non-limiting example, when computing attention scores on the word “am,” decoder should not have access to the word “fine” in “I am fine,” because that word is a future word that was generated after. The word “am” should only have access to itself and the words before it. In some embodiments, this may be accomplished by implementing a look-ahead mask. Look ahead mask is a matrix of the same dimensions as the scaled attention score matrix that is filled with “0s” and negative infinities. For example, the top right triangle portion of look-ahead mask may be filled with negative infinities. Look-ahead mask may be added to scaled attention score matrix to obtain a masked score matrix. Masked score matrix may include scaled attention scores in the lower-left triangle of the matrix and negative infinities in the upper-right triangle of the matrix. Then, when the softmax of this matrix is taken, the negative infinities will be zeroed out; this leaves zero attention scores for “future tokens.” An “attention score,” for the purposes of this disclosure, is a value associated the strength of the relationship between a token or word and other tokens or words in a sequence. In one or more embodiments, an input into a machine learning model may contain a sequence of elements wherein each element may be give a scalar value or ‘attention score’ indicating the importance of each element. In one or more embodiments, attention scores may indicate the relevance or importance of each element within some context of the sequence.

Still referring to FIG. 1, second multi-headed attention layer may use encoder outputs as queries and keys and the outputs from the first multi-headed attention layer as values. This process matches the encoder's input to the decoder's input, allowing the decoder to decide which encoder input is relevant to put a focus on. The output from second multi-headed attention layer may be fed through a pointwise feedforward layer for further processing.

With continued reference to FIG. 1, the output of the pointwise feedforward layer may be fed through a final linear layer. This final linear layer may act as a classifier. This classifier may be as big as the number of classes that you have. For example, if you have 10,000 classes for 10,000 words, the output of that classifier will be of size 10,000. The output of this classifier may be fed into a softmax layer which may serve to produce probability scores between zero and one. The index may be taken of the highest probability score in order to determine a predicted word.

Still referring to FIG. 1, decoder may take this output and add it to the decoder inputs. Decoder may continue decoding until a token is predicted. Decoder may stop decoding once it predicts an end token.

Continuing to refer to FIG. 1, in some embodiment, decoder may be stacked N layers high, with each layer taking in inputs from the encoder and layers before it. Stacking layers may allow an LLM to learn to extract and focus on different combinations of attention from its attention heads.

With continued reference to FIG. 1, an LLM may receive an input. Input may include a string of one or more characters. Inputs may additionally include unstructured data. For example, input may include one or more words, a sentence, a paragraph, a thought, a query, and the like. A “query” for the purposes of the disclosure is a string of characters that poses a question. In some embodiments, input may be received from a user device. User device may be any computing device that is used by a user. As non-limiting examples, user device may include desktops, laptops, smartphones, tablets, and the like. In some embodiments, input may include any set of data associated with electronic health records 120 wherein outputs may include outputs such as identified gastrointestinal diseases or medical diseases.

With continued reference to FIG. 1, an LLM may generate at least one annotation as an output. At least one annotation may be any annotation as described herein. In some embodiments, an LLM may include multiple sets of transformer architecture as described above. Output may include a textual output. A “textual output,” for the purposes of this disclosure is an output comprising a string of one or more characters. Textual output may include, for example, a plurality of annotations for unstructured data. In some embodiments, textual output may include a phrase or sentence identifying the status of a user query. In some embodiments, textual output may include a sentence or plurality of sentences describing a response to a user query. As a non-limiting example, this may include restrictions, timing, advice, dangers, benefits, and the like.

With continued reference to FIG. 1, LLM may be configured to receive inputs such as electronic health records 120 and identify medical factors 128, e.g., gastrointestinal factors, that may be indicative of a medical, e.g., gastrointestinal, disease. In one or more embodiments, LLM may be configured to receive inputs such as electronic health records and identify medical factors that may be indicative of a medical disease. In one or more embodiments, LLM may be configured to extract comments made with physician's notes, patients notes and the like and determine if the notes are correlated to gastrointestinal disease and/or medical disease. In one or more embodiments, LLM may be configured to identify a context of each word or phrase in order to determine if the words or phrases are associated with medical disease. For example, and without limitation, a term such as “stomach pain” may be irrelevant in instances in which a patient has fallen on their stomach. While a force received to the abdomen might indicate that he patient has been hurt it may not indicate a gastrointestinal disease. In one or more embodiments, LLM may be configured to identify entire phrases or statements in order to determine the context of each symptom and/or word within electronic health record 120. In one or more embodiments, LLM may be configured to identify which electronic health record 120 may be associated with a medical disease by identifying information within electronic health records 120 and determining if those statements may be attributed to a medical disease.

With continued reference to FIG. 1, processor 108 may be configured to identify medical factors 128 and/or medical factors such as disease codes, procedure codes, words, phrases and the like and assign medical determination 124 to each electronic health record 120. In one or more embodiments, medical determination 124 may indicate that particular type of gastrointestinal or the lack thereof. For example, and without limitation, a first medical determination 124 may indicate that a patient has BE while a second medical determination 124 may indicate that no gastrointestinal diseases were detected. In one or more embodiments, each electronic health record 120 may be labeled, wherein each label may indicate if a gastrointestinal disease was detected or not detected. In one or more embodiments, each electronic health record 120 may be labeled, wherein each label may indicate if a medical disease was detected or not detected.

With continued reference to FIG. 1, electronic health records 120 may be segmented and/or grouped into separate groupings based on medical determination 124 and/or a gastrointestinal determination. In one or more embodiments, electronic health records 120 may be grouped based on their medical determination 124. For example, and without limitation, electronic health records 120 that have been assigned a label indicating ‘BE’, may be grouped with other electronic health records 120 that have been assigned the same label. Similarly, electronic health records 120 having no label and/or a label indicating that no diagnosis was found may be grouped with other electronic health records 120 in which a gastrointestinal disease was not identified. In one or more embodiments, electronic health records 120 that were labeled with a gastrointestinal disease may be grouped into a ‘gastrointestinal cohort’ while electronic health records 120 that were not labeled with a gastrointestinal disease and/or were indicated not to have gastrointestinal disease may be grouped into a ‘control cohort’. In one or more embodiments, electronic health records 120 that were labeled with a particular medical disease may be grouped into a ‘medical disease positive cohort’ while electronic health records 120 that were not labeled with that medical disease disease and/or were indicated not to have that medical disease may be grouped into a ‘control cohort’. t. In one or more embodiments, the control cohort may include individuals that were screened by processor 108 and determined not to suffer from a gastrointestinal disease. In one or more embodiments, the gastrointestinal cohort may include individuals that were identified to have gastrointestinal diseases based on identified medical factors 128.

With continued reference to FIG. 1, processor 108 may be configured to generate medical training data 132. In one or more embodiments, processor 108 may be configured to generate medical training data. In one or more embodiments, medical training data may include a plurality of electronic health records correlated to a plurality of medical diagnosis. In one or more embodiments, medical diagnosis may be labeled to each electronic heath record similar to that of gastrointestinal determination. In one or more embodiments, medical training data 132 may include any training data as described in this disclosure. In one or more embodiments, gastrointestinal training data may be generated as a function of electronic health records and the presence of gastrointestinal determination. In one or more embodiments, medical training data 132 may be used to train one or more machine learning models. In one or more embodiments, the one or machine learning models may be configured to receive inputs such as electronic health records 120 and output a probability of a gastrointestinal disease and/or a prediction of the patient developing a gastrointestinal disease. In one or more embodiments, computing device 104 may include a machine learning module to implement one or more algorithms or generate one or more machine-learning models to generate outputs. However, the machine learning module is exemplary and may not be necessary to generate one or more machine learning models and perform any machine learning described herein. In one or more embodiments, one or more machine-learning models may be generated using training data. Training data may include inputs and corresponding predetermined outputs so that a machine-learning model may use correlations between the provided exemplary inputs and outputs to develop an algorithm and/or relationship that then allows machine-learning model to determine its own outputs for inputs. Training data may contain correlations that a machine-learning process may use to model relationships between two or more categories of data elements. Exemplary inputs and outputs may come from database 116, user inputs and/or be provided by a user. In other embodiments, a machine-learning module may obtain a training set by querying a communicatively connected database 116 that includes past inputs and outputs. Training data may include inputs from various types of databases 116, resources, libraries, dependencies and/or user inputs and outputs correlated to each of those inputs so that a machine-learning model may determine an output. Correlations may indicate causative and/or predictive links between data, which may be modeled as relationships, such as mathematical relationships, by machine-learning models, as described in further detail below. In one or more embodiments, training data may be formatted and/or organized by categories of data elements by, for example, associating data elements with one or more descriptors corresponding to categories of data elements. As a non-limiting example, training data may include data entered in standardized forms by persons or processes, such that entry of a given data element in a given field in a form may be mapped to one or more descriptors of categories. Elements in training data may be linked to categories by tags, tokens, or other data elements. A machine learning module may be used to create a machine learning model and/or any other machine learning model using training data. Training data may be data sets that have already been converted from raw data whether manually, by machine, or any other method. In some cases, the machine learning model may be trained based on user input. For example, a user may indicate that information that has been output is inaccurate wherein the machine learning model may be trained as a function of the user input. In some cases, the machine learning model may allow for improvements to computing device such as but not limited to improvements relating to comparing data items, the ability to sort efficiently, an increase in accuracy of analytical methods and the like.

With continued reference to FIG. 1, in one or more embodiments, a machine-learning module may be generated using training data. Training data may include inputs and corresponding predetermined outputs so that machine-learning module may use the correlations between the provided exemplary inputs and outputs to develop an algorithm and/or relationship that then allows machine-learning module to determine its own outputs for inputs. Training data may contain correlations that a machine-learning process may use to model relationships between two or more categories of data elements. The exemplary inputs and outputs may come from a database 116, and/or be provided by a user. In other embodiments, machine-learning module may obtain a training set by querying a communicatively connected database 116 that includes past inputs and outputs. Training data may include inputs from various types of databases 116, resources, libraries, dependencies and/or user inputs and outputs correlated to each of those inputs so that a machine-learning module may determine an output. Correlations may indicate causative and/or predictive links between data, which may be modeled as relationships, such as mathematical relationships, by machine-learning processes, as described in further detail below. In one or more embodiments, training data may be formatted and/or organized by categories of data elements by, for example, associating data elements with one or more descriptors corresponding to categories of data elements. As a non-limiting example, training data may include data entered in standardized forms by persons or processes, such that entry of a given data element in a given field in a form may be mapped to one or more descriptors of categories.

With continued reference to FIG. 1, medical training data 132 may include a plurality of electronic health records 120 correlated to a plurality of medical determinations 124. In some embodiments, medical training data 132 may include electronic health records 120 correlated to gastrointestinal diagnoses—this particular embodiment of training data may also be referred to as gastrointestinal training data. In an embodiments, each electronic health record may be associated with one or more medical diagnoses. In an embodiments, medical training data may be used to predict whether patient may develop a medical disease. In one or more embodiments, gastrointestinal cohort (or medical cohort) and/or control cohort may be used to generate medical training data 132. In one or more embodiments, gastrointestinal cohort and control cohort may include training data correlating electronic health records 120 to gastrointestinal determinations 124. For example, and without limitation, electronic health records 120 within gastrointestinal cohort may contain a correlated gastrointestinal determination 124 wherein electronic health records 120 within the control cohort may lack a gastrointestinal determination 124 and/or indicate that the patients lack a gastrointestinal disease. For example, and without limitation, electronic health records 120 within a medical cohort may contain a correlated medical determination 124 wherein electronic health records 120 within the control cohort may lack a particular medical determination 124 and/or indicate that the patients lack a particular medical disease. In one or more embodiments, medical training data 132 may include a plurality of electronic health records 120 correlated to a plurality of medical determinations 124 wherein at least a portion of the plurality of electronic health records 120 lack a medical determination 124. In one or more embodiments, electronic health records 120 within control cohort may lack a medical determination 124 and/or medical disease. In one or more embodiments, medical training data 132 may contain an even amount of electronic health records 120 from the medical cohort and the control cohort. In one or more embodiments, medical training data 132 may contain an uneven balance of electronic health records 120 received from medical cohort or control cohorts. For example, and without limitation, 90% of medical training data 132 may include electronic health records 120 from medical cohort while the remaining 10% may be received from control cohort. In one or more embodiments, medical training data 132 may include ratios of 1:1, 2:1, 3:1, 4:1, 5:1, 6:1, 7:1. 8:1, 9:1 and/or 10:1 of electronic health records 120 received from medical cohorts in comparison to control cohorts. In one or more embodiments, processor 108 may be configured to generate multiple sets of medical training data 132 wherein each set may contain a differing ratio. For example, and without limitation, first set of medical training data 132 may include a 9:1 ratio wherein 90% of medical training data 132 may include electronic health records 120 from the medical cohort while the remaining 10% may include electronic health records 120 from the control cohort. Continuing. a second set of medical training data 132 may include a 4:1 ratio. In one or more embodiments, each set of medical training data 132 may be used to train the same machine learning model wherein outputs of each machine learning model may be compared.

With continued reference to FIG. 1, generating medical training data 132 and/or medical training data may further include identifying a medical history timeframe 136 associated with each electronic health record 120 of the plurality of electronic health records 120. A “medical history timeframe,” for the purposes of this disclosure, refers to a time period within electronic health record 120 in which a patient's past medical information is recorded. For example, and without limitation, the earliest document from electronic health record 120 may be recorded in 2017 and the latest may be recorded in 2023 wherein the medical history timeframe 136 may include a time frame of 6 years. In one or more embodiments, medical history timeframe 136 may denote the recorded medical history of a patient. In one or more embodiments, medical history timeframe 136 may denote the amount of medical information recorded in units of years. For example and without limitation, medical history timeframe 136 may indicate that there are 6 years worth of medical documents within electronic health record 120. In one or more embodiments, medical history timeframe 136 may indicate the earliest date and most recent date in which a patient sought medical treatment. In one or more embodiments, medical history timeframe 136 may indicate the earliest date and the most recent date in which medical information was recorded. In one or more embodiments, electronic health record 120 may contain documents and/or notes in sequential order wherein medical history timeframe 136 may be determined by identifying a date on a first document within electronic health record 120 and identifying a date on a last or most recent document in electronic health record 120. In one or more embodiments, processor 108 may be configured to identify a plurality of dates within electronic health record 120 wherein processor 108 may determine time frame based on a difference between a most recent date and the earliest date identified. In one or more embodiments, electronic health record 120 may contain metadata and/or any other information already indicating medical history timeframe 136. In one or more embodiments, each electronic health record 120 may contain a plurality of documents wherein the documents may indicate a date and time and/or metadata associated with the documents may indicate a date and time of creation.

With continued reference to FIG. 1, in one or more embodiments, processor 108 may be configured to segment plurality of electronic health records 120 based on medical history timeframe 136 and an observation time 140. An “observation time,” for the purposes of this disclosure, refers to an established time frame within electronic health record 120 that has been determined to be of use or importance. In one or more embodiments, only a particular time frame of electronic health record 120 may be of importance wherein observation time 140 may define the established period. In one or more embodiments, observation time 140 may include a period prior to diagnosis and/or identification of medical factor 128. For example, and without limitation, observation time 140 may indicate that only medical information prior to a diagnosis is useful. As a result, processor 108 may segment electronic health record 120 in order to remove any information recorded after the diagnosis. In one or more embodiments, observation time 140 may include a timeframe prior to gastrointestinal determination 124. In one or more embodiments, observation time 140 may include a period of up to 5 or 6 years prior to a gastrointestinal determination 124. For example, and without limitation, in instances in which a diagnosis was made in 2021, processor 108 may be configured to segment electronic health record 120 such that electronic health record 120 only contains medical information spanning from the year 2015/2016 until 2021. All other information may be discarded and/or not of use. In one or more embodiments, observation time 140 may include a timeframe of information that may be of importance when training a machine learning model. In one or more embodiments, observation time 140 may indicate a timeframe of 6 months and/or 1 year prior to a diagnosis until up to 6 years prior to a diagnosis. Observation time 140 may include timeframes beginning at least 1, 2, 3, 4, 5, 6, 7, 8, 9, 10, 11, or 12 month(s) and/or year(s) prior to a diagnosis. For example, and without limitation, in instances in which a diagnosis was made in 2021, the electronic health record 120 may be segmented such that only medical information spanning from 2015 until 2020 are recorded. In one or more embodiments, observation time 140 may include any span of time prior to gastrointestinal determination 124. In an embodiment, a time frame prior to a diagnosis may be used to identify symptoms or factors that led to the diagnosis. In one or more embodiments, observation time 140 may include a span of at least one year prior to a diagnosis to cover instances in which a patient suffered from a gastrointestinal disease but was not yet diagnosed.

With continued reference to FIG. 1, each element, medical document and the like may contain a correlated temporal feature 144. A “temporal feature,” for the purposes of this disclosure, is information indicating the date of creation or recordation of an element within electronic health record. For example, and without limitation, temporal feature 144 may include a date in which a medication was prescribed, a date in which a physician note was written, a date in which laboratory results were received and the like. In one or more embodiments, each element of document within electronic health record 120 may contain a correlated temporal feature 144. In one or more embodiments, database 116 such as patient database 116 may record a time in which each medical document was received and placed into electronic health record 120. In one or more embodiments, each element or medical document may contain a date and/or time in which the document was recorded. In one or more embodiments, metadata may indicate the date and/or time in which a medical document was recorded. In one or more embodiments, electronic health record 120 may include a plurality of data elements. A “data element” for the purposes of this disclosure refers to a unit of information. For example, and without limitation, data element may include a name, an age, a medication taken, a sex of the individual, the race of the individual, the family history of the individual and the like as indicated within electronic health record 120. In one or more embodiments, electronic health record 120 may be made up of a plurality of data elements wherein each data element may refer to a single medical document, a portion of a medical document, a particular set of information and the like. In one or more embodiments, data elements may include medical factors 128, wherein each medical factor 128 may include a separate data element. In one or more embodiments, data elements may refer to medications given, treatments given and the like. In one or more embodiments, data elements may contain corresponding temporal features 144. In one or more embodiment, each data element may be associated with a temporal feature 144. In one or more embodiments, multiple data elements may contain similar temporal features 144, such as for example, data elements extracted from the same document. In one or more embodiments, each data element may correspond to a differing set of information, such as but not limited to, a particular medication given, an age, a diagnosis, past history and the like.

With continued reference to FIG. 1, processor 108 may identify medical factor 128 and/or medical factor and identify a correlated temporal feature 144. In one or more embodiments, medical factor 128 may be identified within a medical document wherein the correlated temporal feature 144 may include the date in which the document was created or recorded. In one or more embodiments, medical factors 128 may contain correlated temporal features 144 wherein the temporal features 144 may originate from the document in which medical factor 128 was identified. In one or more embodiments, processor 108 may determine a date of diagnosis based on medical factor 128 wherein processor 108 may be configured to segment electronic health record 120 based on medical factor 128 and observation time 140. For example, without limitation a diagnostic code identified within a medical document from 2015 may indicate that patient had a gastrointestinal disease. As a result, processor 108 may be configured to segment electronic health record 120 such that electronic health record 120 only contains medical information from prior to 2015. In one or more embodiments, electronic health records 120 originating gastrointestinal cohort may be segmented due to the presence of medical factors 128 whereas electronic health records 120 from control cohort may not be segmented due to the lack of medical factors 128 and/or a lack of gastrointestinal determination 124. In one or more embodiments, control cohorts may instead be segmented to contain the most recent five or six years of electronic health record 120. For example, and without limitation, an electronic health record 120 within control cohort having an observation a time frame from 2015-2024 may be segmented such that only information from 2018/2019 until 2024 may be retained. In one or more embodiments, in instances in which a gastrointestinal determination 124 is not made and/or a medical factor 128 was not identified, observation time 140 may include the most recent date recorded within electronic health record 120 rather than the date of diagnosis. For example, and without limitation an electronic health record 120 having a time frame of 2015-2024, observation time 140 may use the year 2024 to segment electronic health record 120. As a result, electronic health record 120 may be segmented to include only information from 2018/2019 until 2023/2024. In one or more embodiments, electronic health records 120 may be segmented prior to being used in medical training data 132. In one or more embodiments, medical training data 132 may include an observation time 140 indicating a time frame prior to a diagnosis and/or a timeframe prior to the most recent date within electronic health record 120. In one or more embodiments, observation time 140 includes a time frame covering at least one year prior to at least one medical factor 128 of the one or more medical factors 128.

With continued reference to FIG. 1, medical training data 132 may be configured to train one or more gastrointestinal machine learning models. In an embodiments, medical training data may be used to train one or more medical machine learning models. In one or more embodiments, medical machine learning models may receive electronic health records 120 as an input and output a medical diagnosis. In one or more embodiments, medical machine learning model may include medical machine learning model, wherein medical machine learning model 148 is configured for a particular gastrointestinal disease. In an embodiment, each medical machine learning model may be configured to receive an input such as electronic health record 120 and may be trained to produce an output indicating a probability of gastrointestinal determination 124 and/or a probability of gastrointestinal disease. In one or more embodiments, more than one medical machine learning models 148 may be trained wherein each medical machine learning model may be trained on similar training data but having differing ratios between electronic health records 120 from gastrointestinal cohorts and electronic health records 120 from control cohorts. For example, and without limitation, a first medical machine learning model may be trained with a first medical training data 132 set having a 5:1 ratio while a second medical machine learning model may be trained on a second medical training data 132 set having a 9:1 ratio. In one or more embodiments, one or more medical machine learning models 148 may be trained wherein each medical machine learning model may be trained on medical training data 132 having differing ratios of electronic health records 120 from gastrointestinal cohorts and control cohorts.

With continued reference to FIG. 1, medical machine learning model may include parameter values. “Parameter values” for the purposes of this disclosure are internal variables that a machine learning model has generated from training data in order to make predictions. In one or more embodiments, parameter values may be adjusted during training or pretraining in order to minimize a loss function. In one or more embodiments, during training, predicted outputs of the machine learning model are compared to actual outputs wherein the discrepancy between predicted output and actual outputs are measured in order to minimize a loss function. A loss function also known an “error function” may measure the difference between predicted outputs and actual outputs in order to improve the performance of the machine learning model. A loss function may quantify the error margin between a predicted output and an actual output wherein the error margin may be sought to be minimized during the training process. The loss function may allow for minimization of discrepancies between predicted outputs and actual outputs of the machine learning model. In one or more embodiments, the loss function may adjust parameter values of the machine learning model. In one or more embodiments, in a linear regression model, parameter values may include coefficients assigned to each feature and the bias term. In one or more embodiments, in a neural network, parameter values may include weights and biases associated with the connection between neurons or nodes within layers of the network. In one or more embodiments, during training and/or pretraining of the machine learning model, parameter values of the machine learning model may be adjusted based on predicted outputs and a comparison between the predicted outputs and actual outputs. In one or more embodiments, processor 108 may be configured to minimize a loss function by adjusting parameter values of medical machine learning model based on discrepancies between predicted output and actual outputs. In one or more embodiments, a performance of medical machine learning model may be determined using a validation set. A “validation set” for the purposes of this disclosure is a portion of training data that is held from the machine learning model and used to determine the performance of the machine learning model. For example, and without limitation, validation set may include a plurality of electronic health records 120 correlated to medical determinations 124 and/or a lack thereof wherein medical machine learning model may be configured to receive electronic health records 120 and output a predict medical determination 124. In one or more embodiments, outputs of medical machine learning model may be compared to correlated outputs within validation set wherein parameter values of medical machine learning model may be modified based on discrepancies between predicted outputs and actual outputs within validation set. In one or more embodiments, validation set may include electronic health records 120 from gastrointestinal cohorts, medical cohorts and/or electronic health records 120 from control cohorts that have not been used within medical training data 132. In one or more embodiments, validation set may include a portion of medical training data 132 that has not been used to train medical machine learning model. In one or more embodiments, training medical machine learning model may include predicting outputs of validation set and comparing predicted outputs to actual outputs within validation set. For example, and without limitation, medical machine learning model may receive an electronic health record 120 and predict a medical determinations 124 wherein the predicted medical determination 124 may be compared to the actual medical determination 124 within validation set.

With continued reference to FIG. 1 medical machine learning model may include a transformer-based machine learning model 152. A “transformer-based machine learning model,” as described herein, refers to a machine learning model that comprises a transformer architecture. In an embodiment, a machine learning model may process elements in an input independently and/or sequentially while a transformer-based machine learning model may capture dependencies and relationships between inputs using attention mechanisms. For example, and without limitation, in a transformer-based machine learning, the sequence in which the inputs are received may be used to determine an output. In one or more embodiments, transformer-based machine learning model 152 may be used to handle sequential data, such as text by capturing relationships between words or tokens in an input sequence. In one or more embodiments, the attention mechanism 156 in the machine learning model helps the model understand the importance of an element with respect to its placement within a sequence. For example, and without limitation, transformer-based machine learning model 152 may be configured to capture the importance of a first medication given prior to a second medication being given as indicated within an electronic health record 120. In one or more embodiments, transformer-based machine learning models 152 may be used to capture trends within sequences of input data, find anomalies and the like. In one or more embodiments, in the context of medicine, attention mechanisms 156 may be used to identify relationships between elements within electronic health records 120. In one or more embodiments, attention mechanisms 156 may focus on distinct relationships within electronic health records 120, such as but not limited to, medications taken, treatment given, diagnosis given and the like. A transformer-based machine learning model 152 architecture may be used to develop a predictive model such as medical machine learning model. Transformer models may use attention mechanisms 156 to capture the temporal interdependencies of words. Patient characteristics such as symptoms, diagnostic codes, medications, laboratory tests, and demographics within electronic health record 120 may be used in lieu of words. The temporal sequence of all these features may be maintained as they occurred in the patient time frame within electronic health record 120. During model development, the feature and positional vectors may be passed to transformer encoders. The transformer encoders convert the feature and positional vector into an intermediate vector representing a patient's temporal events. This intermediate vector is then combined with nontemporal information to generate comprehensive vector of the patient. Transformer-based machine learning model 152 may be described in further detail below.

With continued reference to FIG. 1, transformer-based machine learning model 152 may utilize an attention mechanism 156 to weigh the importance of words and/or temporal interspecies in a sequence in order to make predictions. In one or more embodiments, the attention mechanism 156 may allow for the model to capture long range dependencies and contextual information. In one or more embodiment, a transformer architecture of the transformer-based machine learning model 152 may include an encoder and a decoder. In one or more embodiments, the encoder may be configured to receive the input sequence and capture contextual information of the input sequence. In one or more embodiments, the encoder may include multiple layers wherein each layer may include a multi-head attention mechanism 156 and a feed-forward neural network. The multi-head attention mechanism 156 allows the model to capture dependencies between words and/or features within inputs and create a weighted representation for each word or feature in the input. In one or more embodiments, the attention mechanism 156 may compute attention weights for each word or feature by considering similarities between other words and features. In one or more embodiments, the attention weights may be used to generate a weighted of the input. In one or more embodiments, the feed-forward neural network processes the representations made by the attention mechanism 156 and applies non-linear transformations on order to capture more complex patterns and relationships. In one or more embodiments, each layer of the encoder may allow for more determinations of representations between inputs. In one or more embodiments, transformer machine learning model may be configured to apply linear transformations to the encoder outputs to predict outputs. During training, the model may learn to adjust parameters of various components (also known herein as “parameter values) such as but not limited to embeddings, attention mechanisms 156, feed-forward networks and the like. In one or more embodiments, transformer-based machine learning model 152 may be trained to predict a probability of a medical (e.g., gastrointestinal) disease. In one or more embodiments, transformer-based machine learning model 152 may be configured to receive inputs such as electronic health records 120 and output a probability of a medical disease. In one or more embodiments, parameter values of the transformer-based machine learning model 152 may be iteratively adjusted in order to make more accurate predictions. In one or more embodiments, parameter values of transformer-based machine learning model 152 ma include embedding parameters. In one or more embodiments, embedding parameters may determine how inputs are initially transformed into numerical representations. In one or more embodiment parameter values may include attention weights. In one or more embodiments, attention weights may determine how much each input may contribute to an output such as a probability of disease. In one or more embodiments, parameter values may include feed-forward neural network weights. In one or more embodiments, feed-forward neural network weights may be used to capture nonlinear relationships between inputs. In one or more embodiments, parameter values may be adjusted to train transformer-based machine learning model 152. In one or more embodiments, parameter values may indicate the importance of various inputs received by transformer-based machine learning model 152.

With continued reference to FIG. 1, transformer-based machine learning model 152 may be configured to use attention mechanisms 156 to capture temporal interdependencies within plurality of electronic health records 120. In one or more embodiments, attention mechanism 156 may include any attention mechanism 156 as described in this disclosure. In one or more embodiments, attention mechanism 156 may be used to capture temporal interdependencies. A “temporal interdependency” for the purposes of this disclosure refers to a time-based relationship between two data elements. For example, and without limitation, temporal interdependency may include a relationship between a two data elements, such as a medication and a diagnosis, wherein the medication was provided following the diagnosis. In one or more embodiments, temporal interferences may be determined by the associated temporal features 144 of each data element. In one or more embodiments, processor 108 and/or transformer-based machine learning model 152 may be configured to capture temporal interdependencies to make one or more determinations. In one or more embodiments, transformer-based machine learning model 152 may identify temporal interdependencies and generate outputs and/or probabilities as a function of the temporal intendencies. In one or more embodiments, temporal interdependencies may contain associated weights and/or parameter values wherein each temporal interdependency may affect an output of transformer-based machine learning model 152. In one or more embodiments, transformer-based machine learning model 152 may be configured to identify data elements, determine temporal features 144 for associated data elements and capture temporal interdependencies as a function of the data elements and the temporal features 144. In one or more embodiments, transformer-based machine learning model 152 may generate outputs based on weights and/or parameter values associated with temporal intendencies. In one or more embodiments, attention mechanism 156 may weigh the importance of each data element in a sequence of data elements relative to one another, wherein attention mechanism 156 may be configured to capture long-range dependencies.

With continued reference to FIG. 1, processor 108 and/or transformer-based machine learning model 152 may be configured to generate attention scores 160. In one or more embodiments, attention scores 160 may include any attention scores 160 as described in this disclosure. In one or more embodiments, attention scores 160 are used to determine the relevance and/or importance of each data element within an input relative to others. In one or more embodiments, attention scores 160 may be used to determine how much focus each input should receive when generating a particular output. In one or more embodiments, attention scores 160 may be generated using a scoring function, such as a dot product or a learned function. In one or more embodiments, attention scores 160 may be normalized across input tokens to obtain attention weight. In one or more embodiment, attention scores 160 allow for transformer-based machine learning model 152 to selectively attend to relevant information within inputs in order to generate more accurate outputs. In one or more embodiments, attention scores 160 allow for transformer-based machine learning model 152 to determine the importance or relevance of each input token. For example, and without limitation, inputs tokens assigned with higher attention scores 160 may be more relevant in the determination of outputs. In one or more embodiments, during training, transformer-based machine learning model 152 may be configured to adjust parameter using task specific objectives, such as prediction of a gastrointestinal disease. In one or more embodiments, during training, transformer-based machine learning model 152 may be trained to associate various input tokens with output tokens based on context provided by other input tokens. In one or more embodiments, the contextual understanding allows the model to assign a higher importance to various input tokens based on their relevance. In one or more embodiments, an attention mechanism 156 may be trained to weigh input tokens based on their relevance or attention scores 160.

With continued reference to FIG. 1, medical machine learning model and/or transformer-based machine learning model 152 may be configured to assign weights to inputs in order to generate outputs. In one or more embodiments, weights may indicate the importance of an input relative to the output. In one or more embodiments, weights may be assigned to various data elements such as medications, treatments, lab results and the like. In one or more embodiments, medical machine learning model may be trained to determine a weighting for each data element wherein medical machine learning model may assign weights when determining outputs. In one or more embodiments, medical machine learning model machine learning model may be configured to generate outputs as a function of temporal features 144. In an embodiment, medical machine learning model may determine relationships between data elements based on temporal features 144. In one or more embodiments, medical machine learning model may determine a relationship between data elements within a sequence. In one or more embodiments, medical machine learning model may be configured to assign a weight to each temporal feature 144 wherein weights may be used to generate outputs of medical machine learning model. In one or more embodiments, weights may be assigned based on a relationship between data elements and/or corresponding temporal features 144.

With continued reference to FIG. 1, medical machine learning model may include transformer-based machine learning model 152, wherein transformer-based machine learning model 152 is configured to capture relationships between inputs and generate a probability of a medical determination 164. The predictive capability of system 100 may include not only a risk prediction described herein, but also a detection of one or more currently undiagnosed conditions. In some embodiments, probability of a medical determination 164 may include a probability of gastrointestinal determination, wherein the probability of gastrointestinal determination is the likelihood that a patient or user suffers from a gastrointestinal disease. As used herein, a “probability of a medical determination” refers to the likelihood in which a user or patient may suffer from, or in the future, may develop, a medical disease. For example, and without limitation, a probability of a medical determination 164 may include 70%, wherein medical machine learning model may predict that there is a 70% likelihood that a patient will be diagnosed with a gastrointestinal disease based on their electronic health record 120. For example, and without limitation, a probability of a medical determination 164 may include 80%, wherein medical machine learning model may predict that there is a 80% likelihood that a patient will be diagnosed with PH-COPD based on their electronic health record 120. In some embodiments, probability of a medical determination 164 may include 90%, wherein medical machine learning model may predict that there is a 90% chance that a patient suffers from PH-COPD. In some embodiments, probability of a medical determination 164 may include a risk prediction for a patient developing BE or EAC. In one or more embodiments, probability of medical determination 164 may include a prediction of whether the patient may suffer from a gastrointestinal disease in the future. In one or more embodiments, a probability of medical determination 164 may include a prediction of the patient developing and/or suffering from a medical (e.g., gastrointestinal) disease in the near future. In one or more embodiments, medical machine learning model may be configured to receive inputs such as electronic health records 120 and output probability of medical determination 124. In one or more embodiments, medical machine learning model may be configured to generate a softmax score. In one or more embodiments, the medical determination 124 may include a softmax score ranging from 0 to 1. A “softmax score” for the purposes of this disclosure refers to a number representing a probability of an output occurring. For example, and without limitation, a machine learning model may generate multiple outputs wherein the softmax score 168 may include the probability of each output occurring. In this instance, softmax score 168 may refer to a probability of a medical determination 164. In one or more embodiments medical machine learning model may output the probability that a patient will be diagnosed with a medical disease and a probability in which the patient will not be diagnosed with the medical disease. The probabilities may each contain values that when added equate to 1. For example, and without limitation, medical machine learning model may output that the probability of a medical determination 164 is 0.8 and the probability that the patient will not be given a diagnosis is 0.2. In one or more embodiments, the probabilities may be calculated using the softmax function which ensures that all probabilities sum up to 1. In one or more embodiments, the softmax score 168 may indicate the probability that the patient will be diagnosed with a medical disease from 0 to 1, wherein a score closed to zero may indicate low confidence in the diagnosis and a score closed to 1 may indicate a higher confidence in the diagnosis. In one or more embodiments, outputs of medical machine learning model may include raw scores, sometimes referred to as ‘logits’ wherein softmax function may receive the raw scores and generate softmax scores 168 ranging from 0 to 1. In one or more embodiments, medical machine learning model may output numerical representations wherein softmax function may be used to convert numerical representations into probabilities.

With continued reference to FIG. 1, processor 108 may be configured to train an ensemble model 172 as a function of one or more medical machine learning models 148. An “ensemble model,” as described herein, refers to a machine learning model in which learned features of multiple machine learning models or outputs of multiple machine learning models are combined to generate a singular output with increased accuracy. For example, without limitation ensemble machine learning model may take an output generate from multiple medical machine learning models 148 and generated a weighted average based on the outputs. In one or more embodiments, the weighted average may be used to determine the probability of gastrointestinal determination or medical determination 124. In one or more embodiments, each medical machine learning model may contain training data having differing ratios between medical cohorts and control cohorts. In one or more embodiments, ensemble model 172 may generate an average of each medical machine learning model to generate accurate results. In one or more embodiments, ensemble model 172 may be configured to determine weightings for each output of each medical machine learning model during training. In an embodiment, training ensemble model 172 may include adjusting weighted averages of each medical machine learning model until a desired output is generated. In one or more embodiments, ensemble model 172 may be trained with a test set that has not been seem by any medical machine learning models 148. In one or more embodiments, learned feature of multiple medical machine learning models 148 may be fed into ensemble model 172. In one or more embodiments, rather than using raw input data to generate learned features, learned presentations from each medical machine learning model may be fed into ensemble model 172. In one or more embodiments, ensemble model 172 may then be trained using the combined learned features. In one or more embodiments, ensemble model 172 may receive learned features as inputs and focus on different aspect or combinations of learned features. In one or more embodiments, predictions of ensemble model 172 may be generated based on learned features fed into ensemble model 172. In one or more embodiments, learned features from each medical machine learning model may differ wherein ensemble model 172 may be configured to receive learned features from each medical machine learning model. In one or more embodiments, ensemble model 172 may be configured to receive outputs of one or more medical machine learning models 148 and output a weighted prediction of the patient developing a medical disease. “Weighted predictions” as described herein refer to an output generated from combining outputs of multiple machine learning models. For example, and without limitation, several medical machine learning models 148 may output a prediction of a patient developing a medical disease as indicated by a medical determination wherein ensemble model may be configured to output a weighted prediction by generating averages, weighted averages and the like. In one or more embodiments, weighted prediction may include outputs of ensemble model 172 wherein ensemble model may generate outputs based on learned features from medical machine learning models. In one or more embodiments, each medical machine learning model may be configured to generate a softmax score wherein ensemble model may include an average of the softmax scores.

With continued reference to FIG. 1, ensemble model 172 and/or medical machine learning model may receive an electronic health record 120 and output a probability of medical determination 164. In one or more embodiments, a probability of a medical determination 164 may include a prediction of a patient developing a medical disease. In one or more embodiments, ensemble model 172 and/or medical machine learning model may be trained using a plurality of electronic health records 120. In one or more embodiments, electronic health records 120 may be identified for medical factors 128 wherein a label may be assigned based on the presence of medical factors 128. In one or more embodiments, processor 108 may be configured to receive electronic health records 120 through a user interface and output probability of medical determination 124 through the user interface.

It is to be understood that the steps and/or process described above may be used in reference to any medical disease and/or diagnosis, such as but not limited to, diabetes, heart arrythmia, cancer, skin cancer, heart disease, chronic obstructive pulmonary diseases including pulmonary hypertension, and the like. In one or more embodiments, the methods and/or process as described in this disclosure with respect to gastrointestinal diseases refers to non-limiting examples. In one or more embodiments processes, steps and/or methods described herein may be used to identify medical diagnoses and generate medical training data correlating a plurality of electronic health records to a plurality of medical diagnosis. In an embodiments, processes, steps and/or methods described herein may be used to predict the development of a medical diagnosis similar to that of probability of gastrointestinal disease 164. In one or more embodiments, ensemble model 172 may be used to generate a weighted prediction of outputs of multiple machine learning models. In one or more embodiments, similar to that of gastrointestinal machine learning model 172, medical machine learning model may be trained to receive electronic health records as inputs and to output predictions of medical diseases. In one or more embodiments, steps and/or processes as described herein are not limited specifically to gastrointestinal diseases, and instead can be used for any medical diagnosis. For example, and without limitation, processor 108 may be configured to identify a diabetic diagnosis using diabetic factors and generate diabetic training data for a diabetic machine learning model. Continuing, the described example may be used to predict the development of diabetes in patients.

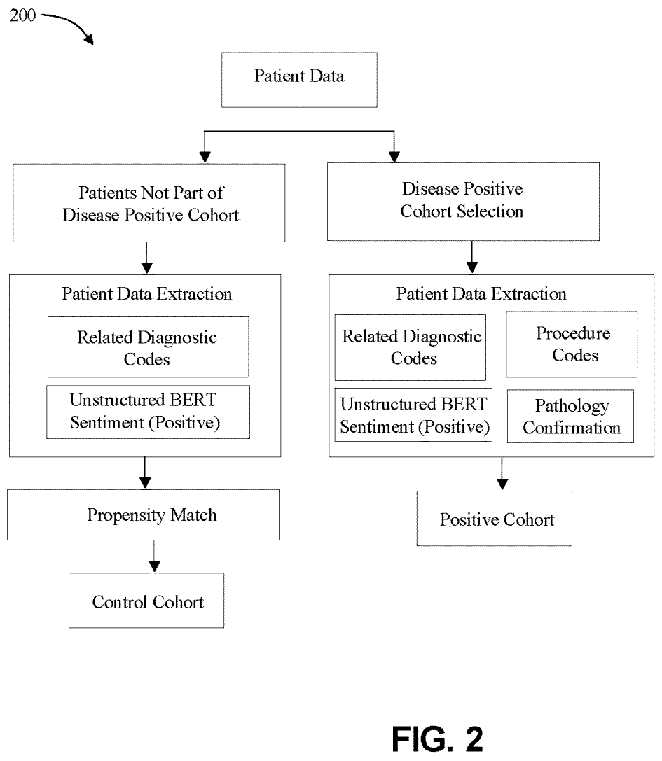

Referring now to FIG. 2, a method 200 for identification of 2 disease cohorts (BE and EAC) is described. In one or more embodiments, four criteria may be used to identify patients included in the BE or EAC disease cohorts. These include but are not limited to (i) diagnosis codes, (ii) endoscopy procedure codes, (iii) augmented curation (an NLP tool), and (iv) the presence of specific keywords in the pathology notes. The International Classification of Diseases (ICD-9 and ICD-10), Systematized Nomenclature of Medicine, and Hospital Adaptation of the International Classification of Diseases codes may be used to identify the diseased cohorts, and the same codes may be used to exclude any cases from the control co-hort. The second criterion may include the presence of an endoscopy procedure code, preceding the diagnosis of BE or EAC. In one or more embodiments, only procedures that are performed within a year of the earliest and latest diagnosis dates may be considered. In one or more embodiments, Patient notes may be processed using models to check whether patients were diagnosed with BE or EAC. This may include a 3-step process which includes identifying synonyms for the disease, getting relevant sentences from the patient notes that mentioned the disease of interest or the synonyms, and using a model to check whether these sentences indicate that the patient had the disease of interest. Various databases, such as MESH terms, DOID, MONDO Ontology, and Wikidata, may be used to identify known synonyms of BE and EAC in the literature. In addition to these tools, manual reading of clinical notes and domain knowledge may be used to come up with terms that identify BE and EAC. For example, “esophageal adenocarcinoma” and “adenocarcinoma of the esophagus” would both be the synonyms for EAC. In one or more embodiments, method 200 may include the identification of sentences from the patient notes that had mention of the disease or its synonyms. In one or more embodiments, a natural language model such as LLM, classification model, Bidirectional Encoder Representations from Transformer may be trained to de-termini whether a sentence indicated that the patient had a disease. The sentences identified from the patient notes may then be processed through this model to check whether the patient was diagnosed with BE or EAC.

With continued reference to FIG. 2, the process of identifying disease-positive patients may require that these patients have, in their pathology notes and/or electronic health records, certain terms related to the disease. For example, the word “adenocarcinoma” along with one of the following other terms—esophageal or esophagus or esophagus or esophageal—may be deemed necessary to confirm a diagnosis of EAC. This may be performed to ensure that the fidelity of anatomical location and pathology is maintained.

With continued reference to FIG. 2, method 200 may include identification of the control cohort and propensity matching to cases. The control cohort may be created by randomly sampling patients from CDAP who did not meet any of the 4 criteria that were used or identification of the disease cohorts. Hence, the patients in the control cohort may not have either any structured or unstructured evidence for BE or EAC. These sampled patients may then be pro-density matched to cases on (i) the year of diagnosis (of the case cohort), (ii) the number of structured disease diagnoses during the observation period (see the definition above), and/or (iii) the proportion of hospitalization in the observation period to the disease cohort (because hospitalization leads to a larger number of medical records per encounter). In one or more embodiments, the cohorts may not be matched to known risk factors of BE/EAC to enable the identification of risk factors agnostic to current knowledge.

With continued reference to FIG. 2, in both the case and control cohorts, patients younger than 18 years and those older than 85 years may be excluded. In addition, only patients who meet the data completeness criteria (defined as having 2 or more encounters in the observation period) may be retained. This may be done to ensure that the model has the op-opportunity to learn from a minimum number of encounters, which optimizes model performance.

With continued reference to FIG. 2, the case identification algorithm described above may be tested against 2 population-based, manually identified, and annotated cohorts of patients with BE and EAC. The cohort may be created using resources from the Rochester Epidemiology Project, which is a population-based medical record linkage system, recently expanded to 11 counties in SE Minnesota.

With continued reference to FIG. 2, in one or more embodiments, the prediction model (i.e., gastrointestinal machine learning model) may be trained on using non temporal features. In one or more embodiments, nontemporal features may include features that do not change with time. In one or more embodiments, nontemporal features may include but are not limited to, age at lead time, sex, race/ethnicity, family history of BE or EAC, smoking status defined as current, past, or never and the like. In one or more embodiments, the prediction model may be trained using temporal features, wherein the temporal features include features associated with time. In one or more embodiments, temporal features may include but are not limited to medications, comorbidities (based on structured analysis) and the like. In one or more embodiments, temporal features may include laboratory tests. Laboratory tests may include tests such as but not limited to hemoglobin, aspartate aminotransferase, alanine aminotransferase, alkaline phosphatase, total bilirubin, albumin, creatinine, sodium, potassium, total cholesterol, low-density lipoprotein cholesterol, high-density lipoprotein cholesterol, triglycerides, chloride, calcium, glucose, blood urea nitrogen, lipase, amylase, gamma glutamyl transferase, prostate-specific antigen, and hemoglobin Alc. These tests may be chosen based on the frequency of occurrence and clinical expertise. In one or more embodiments, temporal features may include symptoms. In one or more embodiments, symptoms may include symptoms identified by augmented curation on patient notes such as but not limited to abdominal pain, dysphagia, dyspepsia, vomiting, diarrhea, heartburn, water brash, chest pain, odynophagia, nausea, snoring, esophageal reflux, dyspnea, arthritis, backache, weight loss, cough, hoarseness, and/or hematemesis. In one or more embodiments, temporal features may further include body mass index of the patient over time.

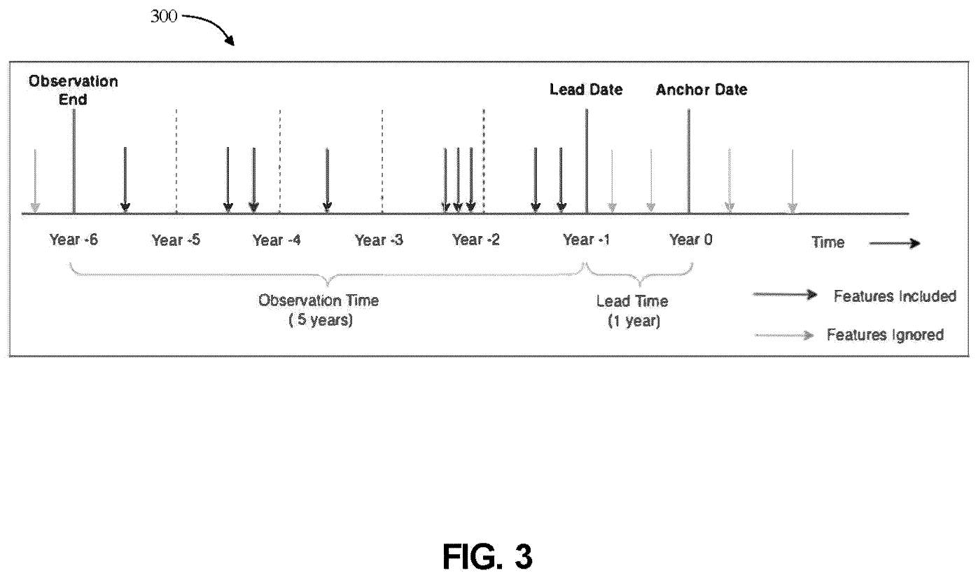

Referring now to FIG. 3, a process 300 of data extraction from the observation time of a hypothetical patient is described. In one or more embodiments, Data from the lead time (1 year before the anchor date: date of initial diagnosis of BE or EAC) may be removed for use in model development, to exclude data which may be reflective of disease symptoms before diagnosis. The observation period (from which data were extracted for model development) may extend from 1 year before diagnosis to 6 years before the diagnosis (i.e., a total of 5 years). A transformer-based ML model architecture may be used to develop the predictive model. Transformer models may use attention mechanisms to capture the temporal interdependencies of words. Patient characteristics such as symptoms, diagnostic codes, medications, laboratory tests, and demographics may be used in lieu of words. The temporal sequence of all these features may be maintained as they occur in a patient timeline. During model development, the feature and positional vectors may be passed to transformer encoders. The transformer encoder may convert the feature and positional vector into an intermediate vector representing a patient's temporal events. This intermediate vector may then be combined with nontemporal information to generate a comprehensive vector of the patient. This vector may then be then passed to a softmax layer to estimate the risk.

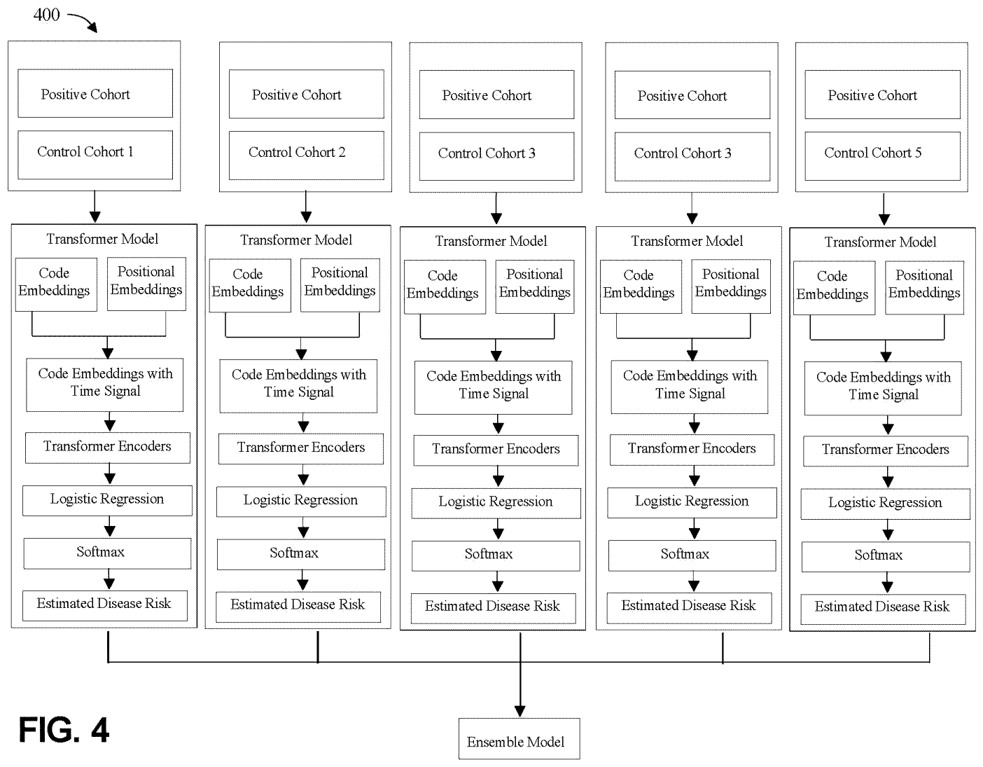

Referring now to FIG. 4, a method 400 for processing a sequence of events in a patient timeline is described. In one or more embodiments, patient timeline may include observation time as described in reference to at least FIG. 1. In one or more embodiments, five randomly selected control cohorts may be created, enabling training of 5 transformer prediction models (or gastrointestinal machine learning models). Each of the transformer models may use the same disease cohort (i.e., gastrointestinal cohort), but trained with a different control cohort. For the BE model, the case-to-control ratio may be 1:5, and for the EAC model, the case-to-control ratio may be 1:10. The output of these 5 transformer models may be used to train an ensemble model using logistic regression. In one or more embodiments, five independent control cohorts may be created. Five control patients may be matched to each patient with BE and 10 control patients matched to each patient with EAC. Five transformer models may be developed by pairing the BE and EAC case cohort with 5 independent control cohorts. These 5 transformer models may then be integrated into a single ensemble model using logistic regression. In one or more embodiments, method 400 may include a schematic showing the layers of the transformer model used to build the BE and EAC machine learning predictive models. BE, Barrett's esophagus; EAC, esophageal adenocarcinoma.

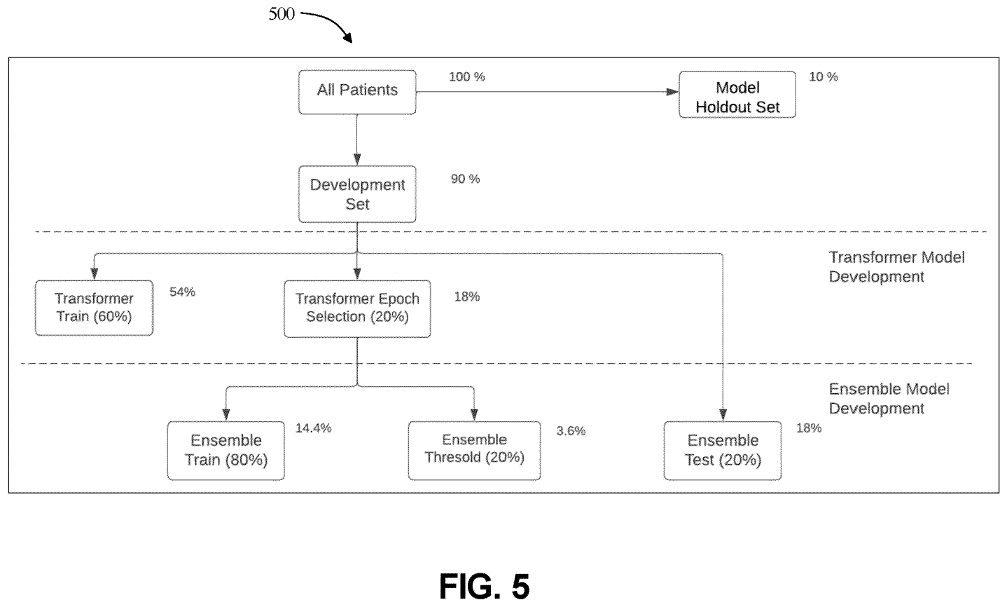

Referring now to FIG. 5, a schematic 500 illustrating the data sets used for a model training process is described. At the outset, 10% of the data may be kept aside as a holdout test data set: the Model Holdout Set (MHS). The rest of the data may be used in training the transformer and ensemble models: the Development Set (DS). The DS may be split into 3 sets in the ratio of 60:20:20. 60% of the DS may be used to train the transformer model, the Transformer Training Set. 20% of the DS may be used to choose the best epoch for the transformer, the Transformer Epoch Set (TES). The last 20% may be used as an Ensemble Test Set. The TES may also be used to train the ensemble model. For this, the TES may be further split in the ratio of 80:20. 80% of the TES may be used for training the ensemble model, the Ensemble Train Set, and 20% may be used to choose to calibrate the ensemble model, the Ensemble Development Set. In one or more embodiments, the development set may be split into 3 ratios of, but not limited to, 70:20:10, 50:30:20, 40-80:20-60:20-60 and the like. The output of the ensemble model may include a softmax score (ranging from 0 to 1, 0 reflecting no risk of BE/EAC and 1 reflecting 100% risk of developing BE/EAC). The threshold for dichotomization for the ensemble result (positive versus negative for incident BE or EAC) may be chosen based on the Youden J method to maximize the area under the receiver-operating curve (AUROC) of the model. A score above the threshold may indicate that the patient is at a substantial risk of being diagnosed with BE or EAC in the next year and screening should be considered.

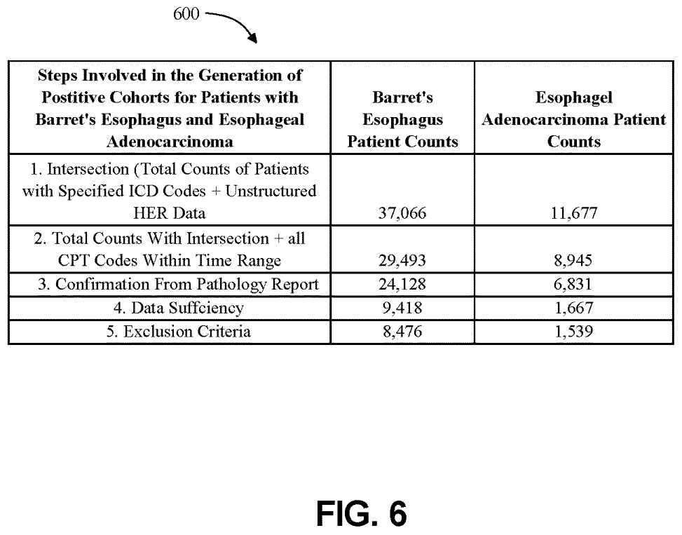

Referring now to FIG. 6, a set of sequential steps taken (as per the inclusion and exclusion criteria described above) and corresponding case counts is described in the form of a table 600. As shown in FIG. 6, table 600 includes a total of 8,476 patients with BE and 1,539 patients with EAC which may be included in the final model development. A total of 252,276 controls may also be identified. Baseline characteristics of the case and control cohorts identified using the electronic search strategy may be presented. Sequential identification of BE and EAC cases from the CDAP, with the application of prespecified data sufficiency, inclusion criteria, and exclusion criteria may be shown in table 600. Most of the BE and EAC cases may include middle aged white men with a past or current history of smoking. Controls may be somewhat younger and more likely to be female than cases. The logic used to generate the case cohorts (BE and EAC) from CDAP may capture approximately 94% of the manually annotated BE and EAC cohorts.

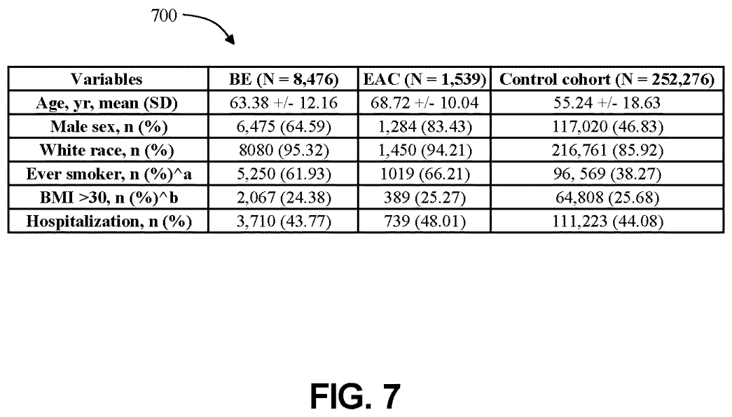

Referring now to FIG. 7, baseline characteristics of the case and control cohorts identified using the electronic search strategy are presented in the form of a table 700. In one or more embodiments, the mean year of patients may include patients that are about 64 years in age with a deviation of plus or minus 12 years. In one or more embodiments, male patients may include 64.59% of the tested group. In one or more embodiments, 95.32% of tested individuals may include white individuals. In one or more embodiments 61.93 of individuals diagnosed with BE as shown in table 700 may include individuals who have smoked in the past. Additional information on characteristics of individuals diagnosed with BE or EAC may be found in table 700.

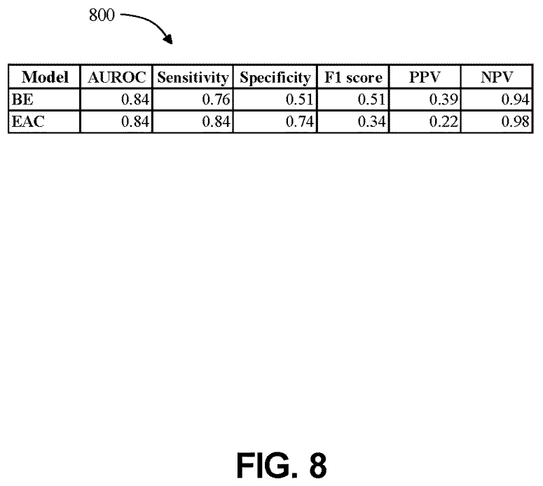

Referring now to FIG. 8, Performance characteristics of the BE and EAC prediction models are presented in the form of a table 800. A threshold of 0.13 (on the model probability output softmax score described earlier) may be chosen to define a positive BE model result. At this threshold, the sensitivity to identify BE may be 76%, at a specificity of 76%, with a model AUROC of 0.84 in the MHS (Table 800). A threshold of 0.08 may be chosen to define a positive EAC model result. In one or more embodiments, the sensitivity for EAC detection may be higher at 84% with a specificity of 61%, with a model AUROC of 0.84 in the MHS (Table 800). Given that this model could be applied to the EHR to first identify those at higher risk of BE/EAC, followed potentially by a minimally invasive non endoscopic test, the threshold to determine positivity for the BE prediction score may be set somewhat lower to balance sensitivity, specificity, and overall AUROC. Conversely, for the EAC threshold, higher sensitivity may be prioritized to avoid missing EAC. Integrated gradients may be used to determine the features that the model used in its prediction. This method attributes a score to each feature for its contribution toward the final outcome. The attribution score for a feature is aggregated across patients. Of note, some determinants (with positive feature scores) increased risk, and some (with negative feature scores) reduced risk. Some of the features that influenced BE risk include male sex, age older than 60 years, ever smoking, gastroesophageal reflux disease (GERD) diagnosis, symptoms of heartburn, dyspepsia, comorbidities such as coronary atherosclerosis, serum triglycerides, and electrolytes. Many of the features that predicted EAC may be similar to those for the prediction of BE (not shown). Notably, a history of BE may be a predictor of incident EAC.

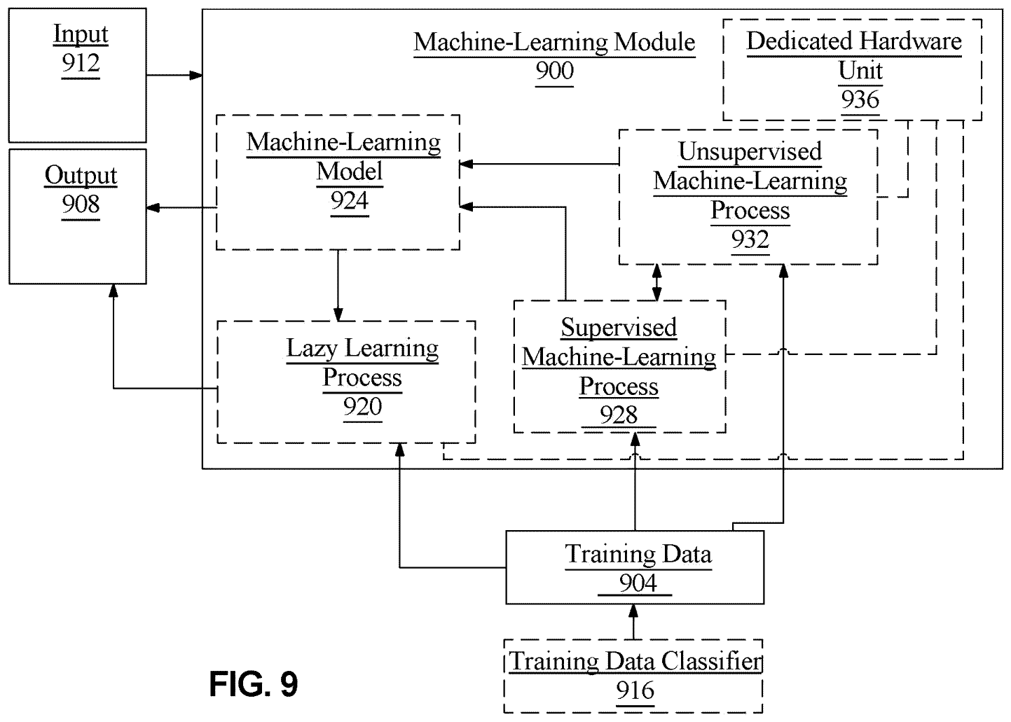

Referring now to FIG. 9, an exemplary embodiment of a machine-learning module 900 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 904 to generate an algorithm instantiated in hardware or software logic, data structures, and/or functions that will be performed by a computing device/module to produce outputs 908 given data provided as inputs 912; this is in contrast to a non-machine learning software program where the commands to be executed are determined in advance by a user and written in a programming language.

Still referring to FIG. 9, “training data,” as used herein, is data containing correlations that a machine-learning process may use to model relationships between two or more categories of data elements. For instance, and without limitation, training data 904 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 904 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 904 according to various correlations; correlations may indicate causative and/or predictive links between categories of data elements, which may be modeled as relationships such as mathematical relationships by machine-learning processes as described in further detail below. Training data 904 may be formatted and/or organized by categories of data elements, for instance by associating data elements with one or more descriptors corresponding to categories of data elements. As a non-limiting example, training data 904 may include data entered in standardized forms by persons or processes, such that entry of a given data element in a given field in a form may be mapped to one or more descriptors of categories. Elements in training data 904 may be linked to descriptors of categories by tags, tokens, or other data elements; for instance, and without limitation, training data 904 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 9, training data 904 may include one or more elements that are not categorized; that is, training data 904 may not be formatted or contain descriptors for some elements of data. Machine-learning algorithms and/or other processes may sort training data 904 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 904 to be made applicable for two or more distinct machine-learning algorithms as described in further detail below. Training data 904 used by machine-learning module 900 may correlate any input data as described in this disclosure to any output data as described in this disclosure. As a non-limiting illustrative example inputs may include inputs such as electronic health records and outputs may include outputs such as gastrointestinal disease predictions.

Further referring to FIG. 9, training data may be filtered, sorted, and/or selected using one or more supervised and/or unsupervised machine-learning processes and/or models as described in further detail below; such models may include without limitation a training data classifier 916. Training data classifier 916 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 900 may generate a classifier using a classification algorithm, defined as a processes whereby a computing device and/or any module and/or component operating thereon derives a classifier from training data 904. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 916 may classify elements of training data to gastrointestinal diseases, wherein each set of training data may be configured to predict the probability of a gastrointestinal disease. For example, and without limitation, a first training data set may be used to predict BE while a second may be used to predict EAC.

Still referring to FIG. 9, Computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. Computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 9, Computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 9, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿaᵢ²}, where aᵢ is attribute number a of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 9, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 9, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 9, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 9, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity, and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 9, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 9, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean upside-effects of compression.

Further referring to FIG. 9, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 9, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset Xₘₐₓ:

$${X}_{n e w} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{n e w} = \frac{X - {X}_{mean}}{{X}_{max} - {X}_{min}} .$$

Feature scaling may include standardization, where a difference between X and Xₘₑₐₙ is divided by a standard deviation σ of a set or subset of values:

$${X}_{n e w} = \frac{X - {X}_{mean}}{σ} .$$

Scaling may be performed using a median value of a set or subset X_{median} and/or interquartile range (IQR), which represents the difference between the 25^{th} percentile value and the 50^{th} percentile value (or closest values thereto by a rounding protocol), such as:

$${X}_{n e w} = \frac{X - {X}_{median}}{IQR} .$$

Persons skilled in the art, upon reviewing the entirety of this disclosure, will be aware of various alternative or additional approaches that may be used for feature scaling.

Further referring to FIG. 9, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 9, machine-learning module 900 may be configured to perform a lazy-learning process 920 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 904. Heuristic may include selecting some number of highest-ranking associations and/or training data 904 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 9, machine-learning processes as described in this disclosure may be used to generate machine-learning models 924. A “machine-learning model,” as used in this disclosure, is a data structure representing and/or instantiating a mathematical and/or algorithmic representation of a relationship between inputs and outputs, as generated using any machine-learning process including without limitation any process as described above and stored in memory; an input is submitted to a machine-learning model 924 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 924 may be generated by creating an artificial neural network, such as a convolutional neural network comprising an input layer of nodes, one or more intermediate layers, and an output layer of nodes. Connections between nodes may be created via the process of “training” the network, in which elements from a training data 904 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 9, machine-learning algorithms may include at least a supervised machine-learning process 928. At least a supervised machine-learning process 928, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include inputs such as electronic health records as described above as inputs, probability of gastrointestinal determination as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 904. Persons skilled in the art, upon reviewing the entirety of this disclosure, will be aware of various possible variations of at least a supervised machine-learning process 928 that may be used to determine relation between inputs and outputs. Supervised machine-learning processes may include classification algorithms as defined above.

With further reference to FIG. 9, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 9, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 9, machine learning processes may include at least an unsupervised machine-learning processes 932. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 932 may not require a response variable; unsupervised processes 932 may be used to find interesting patterns and/or inferences between variables, to determine a degree of correlation between two or more variables, or the like.

Still referring to FIG. 9, machine-learning module 900 may be designed and configured to create a machine-learning model 924 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 9, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 9, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 9, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 9, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 9, one or more processes or algorithms described above may be performed by at least a dedicated hardware unit 936. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 936 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 936 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 936 to perform one or more operations described herein, such as evaluation of model and/or algorithm outputs, one-time or iterative updates to parameters, coefficients, weights, and/or biases, and/or any other operations such as vector and/or matrix operations as described in this disclosure.

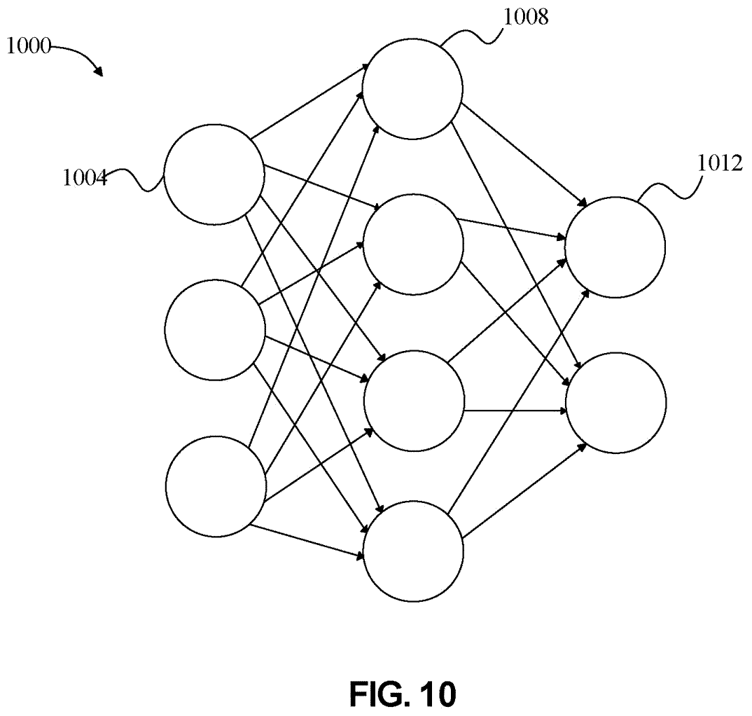

Referring now to FIG. 10, an exemplary embodiment of neural network 1000 is illustrated. A neural network 1000, also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 1004, one or more intermediate layers 1008, and an output layer of nodes 1012. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

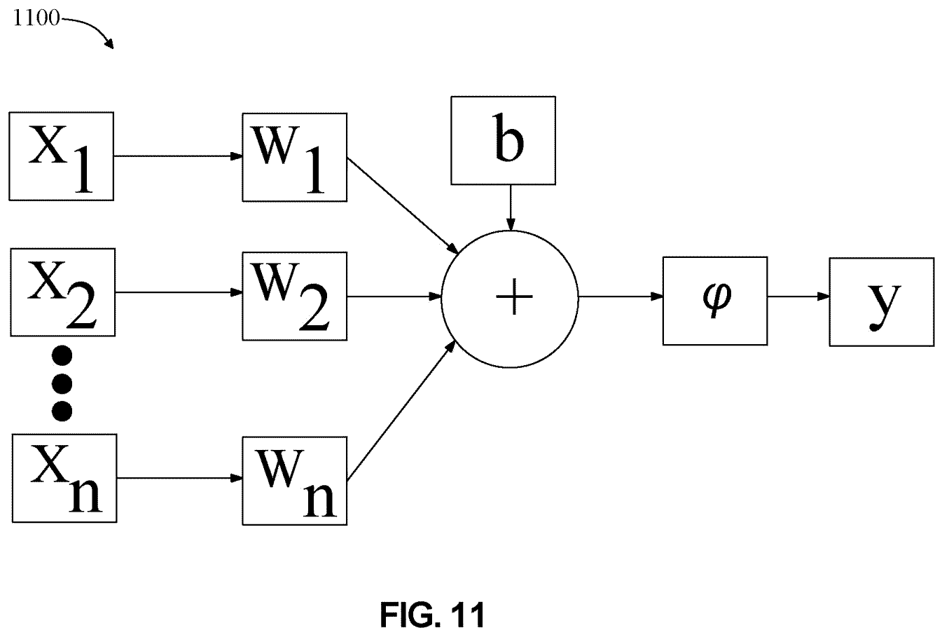

Referring now to FIG. 11, an exemplary embodiment of a node 1100 of a neural network is illustrated. A node may include, without limitation, a plurality of inputs x, that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as ƒ(x)=tanh²(x), a rectified linear unit function such as ƒ(x)=max (0,x), a “leaky” and/or “parametric” rectified linear unit function such as ƒ(x)=max (ax, x) for some a, an exponential linear units function such as

$$f ( x ) = \{ \begin{matrix}x for x \geq 0 \\ α ( {e}^{x} - 1 ) for x < 0\end{matrix}$$

for some value of a (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as ƒ(x)=x*sigmoid (x), a Gaussian error linear unit function such as f(x)=a(1+tanh (\sqrt{2/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

### EXAMPLES

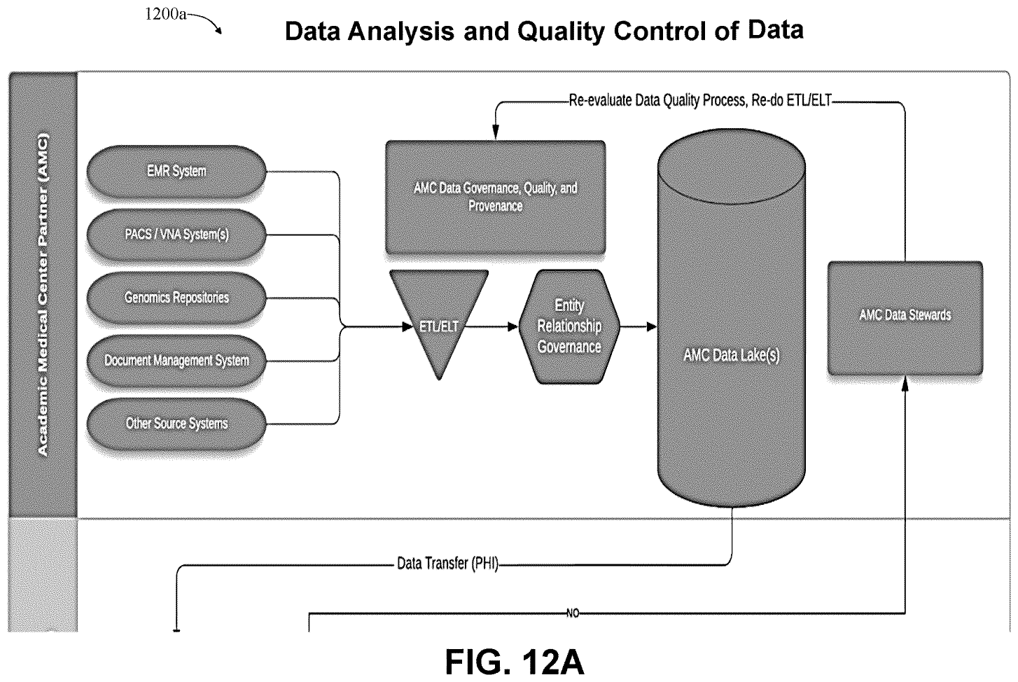

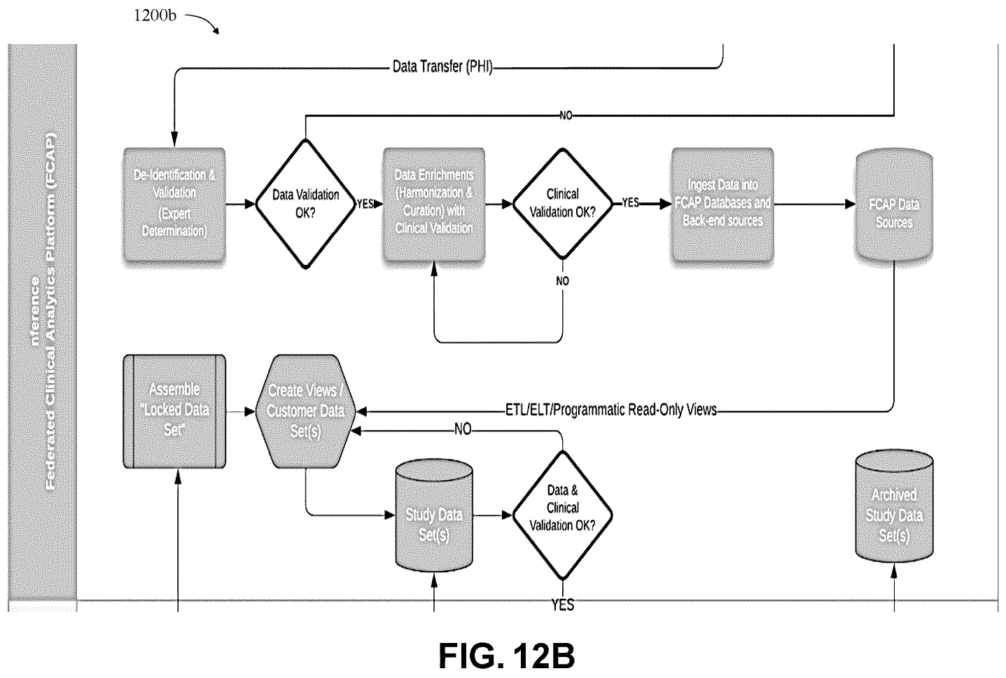

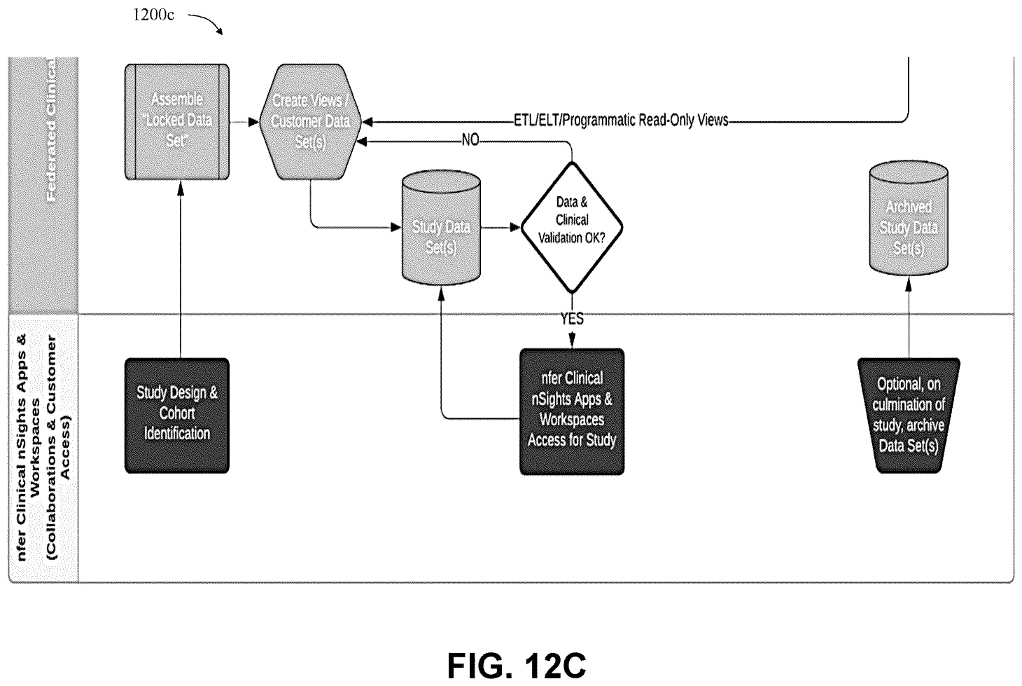

Referring now to FIGS. 12A-C and 13-15, an exemplary study pertaining to system 100 is illustrated.

### 1. ABSTRACT

Title of the Study: characterizing pulmonary hypertension due to chronic obstructive pulmonary disease (PH-COPD) and developing a machine learning (ML) algorithm.

Background/Study Rationale: given the lack of treatments and the need for an invasive procedure to diagnose PH-COPD, it is challenging to identify patients who have PH-COPD and may benefit from a PH-specific therapy. Results from this study were used to develop a machine learning model that can aid in identifying patients likely to have PH-COPD and benefit from therapy. If successful, the model will be validated in other datasets and submitted for qualification by the FDA/EMA to enable its external use.

The objectives of this study are to: i) characterize confirmed PH-COPD diagnosis vs. suspected PH-COPD diagnosis vs. explicitly ruled out PH-COPD diagnosis; ii) develop a preliminary machine learning (ML) algorithm; and iii) estimate prevalence of PH-COPD among patients with COPD across a variety of clinical settings.

Study Design: first, retrospective data may be used for a descriptive analysis of COPD patients using real-world data collected at the Mayo Clinic. Additionally, input from clinicians may be used to ascertain additional patient characteristics that was used to train the algorithm to better identify patients with suspected PH-COPD. Features may also originate directly from clinicians and their experience in treating the condition of interest.

Study Population: Patients with COPD or COPD and PH between 2015-2019

Exposure & Outcome: Correct identification of patients with PH-COPD

Study Setting: This study may use retrospective patient EHR data from the Mayo Clinic.

Statistical Methods: Descriptive statistics for feature space covariates, Cohen's d, t-tests, chi-squared and odds ratios may be performed depending on the type of feature (continuous versus categorical) for a univariate analysis. A multivariate logistic regression analysis may be used to examine the influencing factors between the COPD and PH COPD cohorts. Performance metrics such as AUC, sensitivity, specificity, diagnostic odds ratio, PPV and NPV may be used in algorithm development.

Results: For the four different models (EHR only [supervised], EHR only [self-supervised], ECG only and ECG+EHR), all AUCs were greater than or equal to 0.79, indicating a high level of discriminative ability between COPD patients with and without PH. Based on the three best performing models (EHR only [self-supervised], ECG only and ECG+EHR), the sensitivity-corrected prevalence of PH in COPD populations were as follows: 6.87%, 5.92% and 6.77%, respectively.

Conclusions: The findings of this study indicate that PH-COPD patients have distinct clinical profiles from COPD-only patients and further understanding of this group may help clinicians identify patients who may benefit from PH-COPD screening. The results also show that models may be leveraged in a clinical system to improve detection of PH among COPD patients. This could facilitate PH-COPD patient referrals to PH specialty centers for individualized care in accordance with current guidelines, potentially improving clinical outcomes.

### 2. STUDY OBJECTIVES

This study aimed to identify the key clinical characteristics and patterns suggesting a patient has PH-COPD, as identified by an algorithm. Data regarding patient characteristics may be ascertained via natural language processing (NLP), input from clinicians and machine learning (ML) modeling. Upon completion, results from this project may: (1) curate high quality cohorts of PH-COPD and control patients with complete structured and unstructured clinical records collected in the context of routine clinical care; (2) implement an algorithm for identifying patients with high likelihood of having PH-COPD; (3) evaluate performance of the algorithm in the healthcare system's EMR databases by commonly used metrics (e.g., PPV, NPV, sensitivity, specificity, AUROC, AUPR) or alternative metrics if these cannot be calculated; (4) apply resulting algorithm to large scale unlabeled data from the healthcare system to estimate population prevalence.

### 2.1 Primary Objectives

1). Characterize 3 populations of patients: (1) confirmed PH-COPD diagnosis; (2) suspected PH-COPD diagnosis; and (3) explicitly ruled out PH-COPD diagnosis (e.g., clinical characteristics, echocardiography data, lab values, PFT values, sequence of symptoms). Specifically,
a. Identify clinical characteristics that raise suspicion of PH among COPD patients
b. Describe the patient journey: a descriptive summary of presenting symptoms, referral patterns, diagnostic tests, etc.
2). Develop a preliminary machine learning (ML) algorithm
3). Estimate prevalence of PH-COPD among patients with COPD across a variety of clinical settings (e.g. among all PH patients identified using RHC and Echo values, PH patients with mPAP≥25 versus ≥20, and PH patients with confirmatory RHC data only, combined with the predicted PH-COPD patients from the screening cohort)

### 3. RESEARCH METHODS

### 3.1 Study Design

Descriptive analysis of COPD patients may be performed using structured real-world data collected from the EHR at the Mayo Clinic. Simultaneously, clinical characteristics that raise suspicion of PH among COPD patients may be received from clinicians. A machine learning algorithm may be trained (as described in Section 3.3.3) to identify undiagnosed PH patients more easily in the clinic. For this study, the feature space may consist of covariates from both structured and unstructured EHR data sources including demographics, comorbid diagnoses, lab test results, and measurements collected during ECHO, RHC, ECG, and other procedures. Performance was evaluated using metrics such as area under the curve (AUC), sensitivity and specificity.

### 3.3 Study Population

The populations of interest may include all patients who are confirmed or strongly suspected of PH-COPD and are diagnosed between 2015 and 2019. These dates were chosen because they capture modern treatment practices, but are not impacted by the COVID pandemic, which may have significantly impacted RWE treatment patterns and observed symptoms and associated cardiac and pulmonary conditions. Patients may be selected into the study if they fulfilled all the inclusion criteria and none of the exclusion criteria outlined below. The study population may include three cohorts of patients: a negative cohort (i.e., COPD-only), positive cohort (i.e., PH-COPD) and an unconfirmed screening PH COPD cohort (i.e. COPD patients without RHC or Echo data confirming or ruling-out PH).

The index date for inclusion in the analysis may be defined as: PH diagnosis date. In the PH-COPD cohort, EHR data in predefined temporal windows relative to PH diagnosis date was used. In the COPD-only cohort, EHR data in predefined temporal windows prior to the rule-out echo/RHC was used.

Section Sixty (60) patient IDs from each of these cohorts may be randomly selected and manually reviewed by blinded clinical scientists. The process for patient review may include the following steps:
Random selection of de-identified patients from the positive, negative, and screening cohorts
The clinical scientists were blinded to the generated cohort labels.
The number of patients with each investigator-reviewed label was compared against the generated labels from the platform.
Decision-rules around re-stratifying cohorts were defined based upon level of agreement and need for additional chart reviews.

### 3.3.1 Inclusion Criteria

All inclusion and exclusion criteria may be reviewed by the investigator or qualified designee to ensure that the subject qualifies for the study.

Structured codes to identify patients with COPD at the Mayo Clinic, as well as other information from the electronic medical record may be used to define three populations, as described below:
1). PH-COPD diagnosis.
Patients diagnosed with PH-COPD, defined as:
   Either:
      mPAP≥25 mmHg (eligible for FDA-approved PH treatment)
      mPAP≥20 mmHg (reflects latest ESC/ERS guidelines for PH diagnosis), or
      mPAP≥20 mmHg or if no RHC data available, TRV>3.4 m/s (confirmed PH via RHC plus high likelihood of PH by echo)
         Note, all measurements may be taken at baseline, i.e. at rest and not during challenge. PH diagnosis date may be defined as the earliest date at which either mPAP or TRV exceeded the specified threshold
Presence of one (1) of the following:
   ICD code for COPD preceding PH diagnosis date or within 3 months following PH diagnosis
   Positive sentiment (“confirmatory language”) for COPD preceding PH diagnosis from notes using NLP or within 3 months following PH diagnosis.
      For example, a note on Jul. 23, 2009: “Patient was diagnosed with COPD”, which would occur before a PH diagnosis by RHC on Oct. 2, 2012.
   and does not have Group 1 (Pulmonary Arterial Hypertension [“PAH”]), Group 2, or Group 4 PH, as defined in section 3.3.2.
2). Explicitly ruled out PH-COPD diagnosis
COPD-Only (i.e., without a Diagnosis of PH), Confirmed Via RHC:
   All RHCs have mPAP≤20 mmHg and no TRV>3.4 m/s prior to last RHC OR all TRV≤2.8 m/s by Transthoracic Echocardiogram if no RHC was performed
   Presence of one (1) of the following:
      ICD code for COPD
      Positive sentiment (“confirmatory language”) for COPD from notes
3). COPD patients with unconfirmed PH status
   Screening cohort (Has COPD, does not have RHC or Echo)
      Has either structured diagnosis or confirmatory language for COPD, and
      Is not in any of the exclusion cohorts listed in section 3.3.2 below.

### 3.3.2 Exclusion Criteria

All Cohorts:
Patient is <18 yo at time of diagnosis
Patient has retracted standing research authorization agreement with Mayo Clinic

And does not have Group 1 (PAH), Group 2, or Group 4 PH, defined as:
1). Group 1 (PAH): patients may be required to meet all criteria to be considered Group 1.
mPAP≥20 mmHg at baseline. Baseline refers to the phase of the RHC itself when the patient is at rest (not during exercise) and has not been administered a drug challenge of any kind. Note, this definition is according to the latest ESC/ERS guidelines for PH diagnosis
Limit to precapillary patients:
   PVR≥2.0 WU
   PCWP≤15 mmHg
Use of at least one (1) PAH medication at any time
   Medication list: Ambrisentan, Ambrisentan, Bosentan, Epoprostenol, Iloprost, Macitentan, Selexipag, Sildenafil, Treprostinil, Tadalafil
2). Group 2 PH: patients may be required to meet all criteria to be considered Group 2.
mPAP≥20 mmHg at baseline
Presence of one (1) of the following for the Group 2 diseases of interest (left ventricular systolic or diastolic dysfunction, valvular heart disease, left heart inflow and outflow obstructions not due to valvular disease and congenital cardiomyopathies)
ICD code for disease(s) of interest within 3 months of PH diagnosis (date of mPAP≥20 mmHg)
Positive sentiment (“confirmatory language”) for disease(s) of interest within 3 months PH diagnosis or PH secondary to disease(s) of interest from notes using NLP
3). Group 4 PH: patients may be required to meet all criteria to be considered Group 4.
   mPAP≥20 mmHg at baseline
Limit to precapillary patients:
   PVR≥2.0 WU
   PCWP≤15 mmHg
Presence of one (1) of the following:
   ICD code for CTEPH (date of mPAP≥20 mmHg)
   Positive sentiment (“confirmatory language”) for CTEPH from notes using NLP.

### 3.3.3 Development of PH Detection Algorithm

The algorithm's feature space contained data elements from both structured and unstructured sources. Based on the feature space fill rates, positive and negative cohorts may be refined, and positive and negative cohorts may be randomized into respective algorithm training and test sets.

Once the algorithm was trained, the performance may be evaluated using analyses described in Section 4.2.1 on its corresponding test set.

When training the algorithm, the following may be considered to improve acceptability by users:
Whether algorithm inputs may contain structured data that can be automatically extracted from the EHR system.
Whether certain features may require manual review of patient charts.
Whether a variable may require a significant number of patients to undergo an additional laboratory test or other procedure and how easy/difficult it is to do that test or procedure. For example, NT-proBNP may not be routinely collected in clinical practice but may be an easily obtained lab test. Similarly, PFTs may not be collected/recorded regularly in all patients but are extremely easy to obtain.

### 3.4 Setting

This study may use retrospective EHR data from the Mayo Clinic Health System (Minnesota, Florida, Arizona sites and all community centers) between 2015-2019. No contingency measures were implemented to manage study conduct because of the pandemic.

### 3.5 Outcomes of Interest

This study has no health outcomes. Our outcome of interest may be the correct identification of patients with PH-COPD, as identified with the algorithm developed using the definitions listed above in Section 3.3.1

### 3.6 Description of Covariates

The objective of this algorithm may be to determine what combination of salient covariates distinguish PH-COPD patients from COPD-only controls to identify undiagnosed patients more easily in the clinic. For this study, the feature space may consist of covariates including demographics, structured and unstructured diagnoses, lab tests, and measurements collected during ECHO, RHC, ECG, and/or other procedures. To maximize the degree of coverage across all covariates, there may be different temporal windows preceding PH or following COPD diagnosis (e.g., 3 months, 6 months, 9 months, 1 year, etc.). The PH diagnosis may be used as the index date, so the time windows prior to PH diagnosis but following COPD diagnosis may be considered. Because the intended use population of the algorithm is patients with COPD, patients diagnosed with PH on the same date as COPD may not be used for training because there's no time window with data at which they have a sole COPD diagnosis without concurrent PH diagnosis and thus this algorithm would not be applicable.

In cases where data coverage for a specific feature was <15% of the cohort, the associated data type was dropped.

To characterize the study cohorts, the following features may be assessed
1). Demographics
Age at diagnosis
Race/ethnicity
Gender
Record longitudinally
   Time from first record to COPD diagnosis
   Time from COPD diagnosis to last record at Mayo
Death
   N (%) patients who have since died
   Time from COPD diagnosis to death
2). Observations
a. Heart rate
b. Blood pressure
c. Height
d. Weight
e. Smoking status
f. Alcohol use
g. Exercise
3). ECG:
a. QT interval
b. P-wave
c. QRS durations
4). Echo (as available):
a. TRV-tricuspid regurgitation velocity
b. IVC (inferior vena cava) diameter
c. TAPSE-tricuspid annular plane systolic excursion
d. RVFAC-right ventricular fractional area change
e. RVSP-right ventricular systolic pressure
f. RV/LV ratio-right ventricular to left ventricular diameter ratio
g. RV (right ventricular) size/mass/wall thickness
h. RV strain
i. PE-pulmonary embolism
j. SV-stroke volume (derived, include method of acquisition)
k. EF-ejection fraction (derived, include method of acquisition)
l. Myocardial performance index (aka Tei index)
m. RA (right atrium) and LA (left atrium) size
n. RVOT VTI-Right ventricular outflow tract velocity time integral
o. PASP-pulmonary artery systolic pressure
p. LVEDV-left ventricular end-diastolic volume
q. LVEF-left ventricular ejection fraction
r. LV (left ventricular) mass
s. sPAP-systolic pulmonary arterial pressure
5). Laboratory tests:
a. BNP-brain natriuretic peptide
b. NT-proBNP-N-terminal pro b-type natriuretic peptide
c. eGFR-Estimated glomerular filtration rate
d. Creatinine
e. Uric Acid
f. Anemia
   i. Hemoglobin
   ii. Hematocrit
   iii. Red blood counts
g. Iron
   i. Serum iron
   ii. Serum ferritin
   iii. Serum transferrin
   iv. Transferrin saturation
h. Sodium
i. Red blood cell width distribution
6). Medication history
a. Diuretics
b. Inhalers
c. Bronchodilators
d. COPD medications including Phosphodiesterase-4 inhibitors, Theophylline, and oral steroids.
7). Other procedures/tests
a. CT scan: lung/chest CT scan, cardiac CT
b. VQ scan
c. X-ray: lung/chest x-ray
d. Pulmonary function test
e. 6-minute walk test
f. Oxygen use
8). Healthcare resource utilization
a. Days hospitalized
b. Emergency department visits
c. Visits to Mayo
d. Healthcare provider specialty (if available at desired level of specificity)
e. Lung or Heart/lung Transplant procedure
f. Other procedures/tests
9). Data from unstructured clinical notes using the disease diagnosis model:
a. Symptoms (both presence and noted absence):
   i. Dyspnea
   ii. Chest pain
   iii. Edema of lower limbs
   iv. Fatigue
   v. Dizziness
   vi. Fainting
   vii. Heart palpitations
   viii. Cyanosis

As inputs to the algorithm, symptoms from unstructured clinical notes in the patients' EHRs may be extracted using the disease diagnosis Augmented Curation model. The neural network used to perform disease diagnosis classification may be initially trained using 18,490 sentences containing nearly 250 different cardiovascular, pulmonary, and metabolic diseases and phenotypes. Each sentence may be manually classified into one of four categories: ‘Yes’ (confirmed phenotype), ‘No’ (ruled out phenotype), ‘Maybe’ (suspected phenotype), and ‘Other’ (alternate context, e.g., family history of a phenotype, risk of adverse event from medication, etc.). Using a 90%: 10% train: test split, the model achieved 93.6% overall accuracy and a precision and recall of 95% or better for both positive and negative sentiment classification.

### 3.7 Study Procedures

### 3.7.1 Ethics Review

The database used within the platform may contain only de-identified patient information (i.e., does not include names, addresses, social security or medical record numbers or other obvious identifiers), and is fully compliant with the HIPAA. Therefore, no ethics review was necessary. Confidentiality of patient records were maintained at all times. All study reports contained aggregate data only and did not identify individual patients. At no time during the study did the sponsor receive patient identifying information.

### 3.7.2 Subject Information and Informed Consent

This study required informed consent.

Inclusion in this study may be in accordance with their standing research authorization agreement with Mayo Clinic. If a patient retracts said authorization, they are automatically removed from the database and will not appear in any analyses.

This study may require IRB/EC review.

Investigators may ensure that personal identifiers were removed from any study files that are accessible to non-study personnel in accordance with applicable laws and regulations.

Whenever feasible, study files may be coded and stripped of personal identifiers, and code keys may be stored separate from study files.

The participant was allowed as much time as wished to consider the information, and the opportunity to question the investigator or other independent parties to decide whether they would participate in the study. Electronic ICF was then obtained using the participant's dated signature and the dated signature of the person who presented and obtained the ICF. The person who obtained the consent may be suitably qualified and experienced and have been authorized to do so by the Chief/Principal Investigator. A digital copy of the signed Informed Consent was given to the participant. The original signed electronic form was retained.

### 3.8 Data Handling and Validation/Data Management/Data Quality Assurance

All data collected for the study was recorded accurately, promptly, and legibly. The investigator or qualified designee was responsible for recording and verifying the accuracy of subject data. By signing this protocol, the investigator acknowledges that his/her electronic signature is the legally binding equivalent of a written signature. By entering his/her electronic signature, the investigator confirms that all recorded data have been verified as accurate.

If this study has been outsourced, the institutional policies of the supplier should be followed for development of data management plans. However, the supplier should ensure compliance with Good Pharmacoepidemiology Practice, and all applicable federal, state, and local laws, rules and regulations relating to the conduct of the study.

Data within the clinical platform may go through several transformations before it is made available to scientists and customers—the image above illustrates the high-level processes of data analysis and quality control of data, beginning with the Academic Medical Center (AMC) Partners, which is the source of all our data (FIGS. 12A-C, embodiments 1200a-c). The platform may leverage the Data Governance frameworks of our AMC Partners to ensure that the quality and provenance of source data is validated for clinical and referential integrity prior to ingestion into the Federated Clinical Analytics Platform (FCAP). Upon ingestion into the FCAP, a team of experts may perform several functions, including de-identification of structured and unstructured data. The de-identification process may be a rigorous, expert determination-based process (details outlined further below), and this process may also involve validation of referential integrity within the data (beyond that of the source AMC's Data Governance process—this is because the platform performs cross-modality linking of clinical data from multiple sources, so cross-table and cross-database referential integrity validation may be a key component). In the event data quality/validation issues are encountered, the platform may reconvene with the Data Stewards of the AMC, so that they can address these issues upstream, and re-process the data extraction and delivery to the FCAP.

Following de-identification, data may undergo several enrichments within the FCAP, namely harmonization and augmented curation (details outlined below), and each of these steps may require rigorous data validation-both from a clinical perspective as well as from a data integrity perspective. The data validation steps during the enrichment phases may be in-line and occur in parallel with the enrichment processes.

Once the enriched data is validated, it may be made available by various means for downstream analytics and data science consumption—specifically, the FCAP may provide access to this data via certain applications and workspaces.

For end-users (data scientists, clinical scientists, etc.) working with the data using certain applications and workspaces, the Schema Visualizer application may provide detailed schema information of the transformed data, including entity relationships as well as provenance and transformation related transparencies, so the user can be sure of the treatment of each individual data element. Details of variable composition, model performance and validation, and more may be made available and displayed for end users. Through this, users may also have the tools and information to be able to determine whether the variables are suited for their use. Additionally, release notes may be made available within the product itself for all users with access as part of the product homepage.

These processes and the included data quality checks are outlined in detail below.

### 3.8.1 De-identification

The platform may use a “hiding-in-plain-sight” expert-certified de-identification process. Our approach has been published in a peer-reviewed publication. From a performance standpoint, our approach may outperform existing tools, with a recall of 0.992 and precision of 0.979 on the i2b2 2014 dataset and a recall of 0.994 and precision of 0.967 on a dataset of 10,000 notes from the Mayo Clinic.

As previously mentioned, one aspect of the de-identification process may be to ensure referential integrity within and across multiple data sources, such as:
Correct patient data mapping (data associated with ID is data associated with hashID)
Consistency of data substitutions and transformations
General data field mapping (e.g. are flowsheets going to corresponding flowsheets)
Specific data field mapping (e.g. are patient ID flowsheets going to patient hashID flowsheets)

### 3.8.2 Harmonization

Harmonization is the process of transforming structured and semi-structured data into unified concepts or variables to enable efficient and comprehensive downstream analysis.

This may include:
Automated aggregation of similar concepts based on data characteristics suggested for review (e.g. the shape of the distribution of a set of measurements being similar)
Mapping of equivalent terms, equivalent ‘entities’ (e.g. lab tests, medications) to a parsimonious dictionary (“deduplication”)
Elimination of data elements (terms, values, entities) that are invalid (e.g. physiologically implausible) based on clinical review
Creation and maintenance of a relational structure among all data elements in the refined dictionary

Harmonization of structured quantitative data (e.g. lab tests) involves technology and software-enabled transformation of data variables, which then may undergo final review and approval by clinical scientists. Any applied transformations may be recorded through the software to track the origins of data and the composition of the final harmonized variables.

Harmonization of structured categorical variables (ex. medications administered, ICD diagnoses) may utilize pipelines that rely on a combination of knowledge graph and logic provided by clinical scientists.

Data harmonization may be an ongoing process. If a variable has been missed, misclassified, or mistransformed, it may be reviewed and updated by our clinical science team. Once validated, the updates are part of a versioned update which is synchronously across setups. The process of harmonization is anchored in four guiding principles. To enable accountability for this dataset processing and earn trust from downstream data users, harmonization may be: Consistent, Clinically informed, Transparent, and/or Well-documented

To attain consistency, we have developed a standardized pipeline through which each patient metric is processed from its many raw forms into a single shared encoding. To ensure that within this pipeline each decision is made in a clinically informed manner, our harmonization is conducted exclusively by clinically trained individuals (either a medical student or a medical professional). To accomplish transparency in harmonization, the handling of each metric within our pipeline is documented and available to all data users. This documentation reveals all raw data forms that were gathered and unified, as well as all instances where values were converted from one unit of measure to another. Annotations are authored by our curators when complex data handling arises (e.g. when outright mislabeling is discovered in the raw data).

Data that does not undergo harmonization is exposed in unharmonized form as it stands following the de-identification process.

### 3.8.3 Augmented Curation

Augmented curation is the process of developing and deploying language-based models to extract sentiment from unstructured text. These models may allow us to transform unstructured data into structured form through extraction of key sentiments and relationships from free text at scale. This may allow downstream users to identify and select patients based on information contained in written text of patients' records in an automated way.

Models may fall into two categories-Base (disease-agnostic, should apply to all patients) or Disease/Therapeutic Area-Specific (would apply to only a subset of patients).

Curation models may leverage Bidirectional Encoder Representations from Transformers (BERT)-based neural networks. BERT models were developed by Google as a pre-trained language model. For the model to perform a specific classification of interest well, we may need to fine-tune this base model using labeled datasets. These datasets may consist of fragments from clinical text with the concepts of interest highlighted.

Datasets are labeled by three independent clinical scientists. Datasets are used either for training or for testing of the model (the same dataset is never used for both). Scientists are provided with a tagging guide including examples and must pass a certification dataset to ensure they understand the model objectives and label definitions before they are certified to label datasets for a new model, ensuring some level of standardized understanding.

Dataset generation strategy may include ensuring diverse representation of concepts relevant to the model (ex. diseases from different therapeutic areas) and sentence structures. Vector embeddings of text fragments that meet the criteria required for the model may be generated and clustered, and datasets may be sampled and created from this sentence universe to generate, train, test and validation datasets which are representative of the diversity of text within the entirety of the clinical notes, where the models may ultimately be deployed. Models may be iteratively trained and tested as the datasets are labeled. Active learning methods may be utilized to sample from the sentence universe and iteratively address failure modes of the model.

Once models reach the desired sentence-level performance (typically at least 0.9 precision and recall on test and validation datasets), they may be deployed across all clinical text for the relevant patient population (all patients for base models). The relevant sentences may be identified using the platform's entity extraction service. For example, for the disease diagnosis model, we may first identify all mentions of diseases in notes, select those sentences, and pass each sentence to the model to get a classification of diagnosis versus not. Note, this may be a computationally intensive and time-intensive process to run. The knowledge graph, built on a combination of public ontologies and models, powers the identification of those concepts in the clinical text.

Once deployed, model performance may be continuously monitored. Users can submit error reports to the team responsible for owning the model. Systematic errors in model performance that are identified may be addressed using active learning to select sentences like those which are causing the error, which may then be tagged and then incorporated into model training and testing. Model evaluation and training may be iterative. Full model may run with all available models that take place every quarter and are part of versioned data releases. This may include “first-time” models which have met performance requirements, as well as improved versions of existing or previously deployed models. All models may be included in each quarterly run. If technical errors are identified in model deployment, a versioned patch release may be made.

### 3.8.4 Study Set Validation

When datasets are created for a defined cohort of interest for a study, there may be additional data quality checks applied. This may include dataset-specific validation of curation models. This may be supported by generating additional labeled datasets for a sampled subset from the dataset. Patient-level validation may also be performed through individual chart review of a sample of patients from each dataset. By default, once study design is completed, the dataset for the study cohort is frozen. Upon request and if appropriate for the study, new records can be released to the dataset based on recent updates. The net new data may go through the same validation and quality check processes described above prior to release.

Once the study was conducted, an independent scientist who was not involved in running the initial study may perform a code review of all the components that were used in the study. This may serve as a secondary validation of the approach and methods used in the study and ensure a level of reproducibility by a second independent scientist. If the study necessitates it, additional reviews can be put in place as requested.

As discussed above, the database used within the platform may contain only de-identified patient information (i.e., does not include names, addresses, social security or medical record numbers or other obvious identifiers), and may be fully compliant with the HIPAA.

Therefore, no ethics review was necessary. Confidentiality of patient records was maintained at all times. All study reports contained aggregate data only and did not identify individual patients. At no time during the study did the sponsor receive patient identifying information.

### 3.9 Changes in the Conduct of the Study

There were no changes in the conduct of the study due to the COVID-19 pandemic.

### 4. STATISTICAL AND ANALYTICAL METHODS

There were no changes in the planned analyses of the study due to the COVID19 pandemic.

### 4.1 Characterize Patient Cohorts

All features were extracted from structured and unstructured data sources.

Descriptive analyses were performed to evaluate patient characteristics in the PH-COPD, COPD-only and screening cohorts. The aggregate number N (%) of patients within four 3-month bins to capture the presence of the following features in each time window may be presented:
Demographics
Lab tests and other observations
Medication history
Comorbidities
ECG findings
Echo findings
Imaging findings

Means, SD, median and IQR was reported for continuous variables, and frequencies and percentages may be reported for categorical variables.

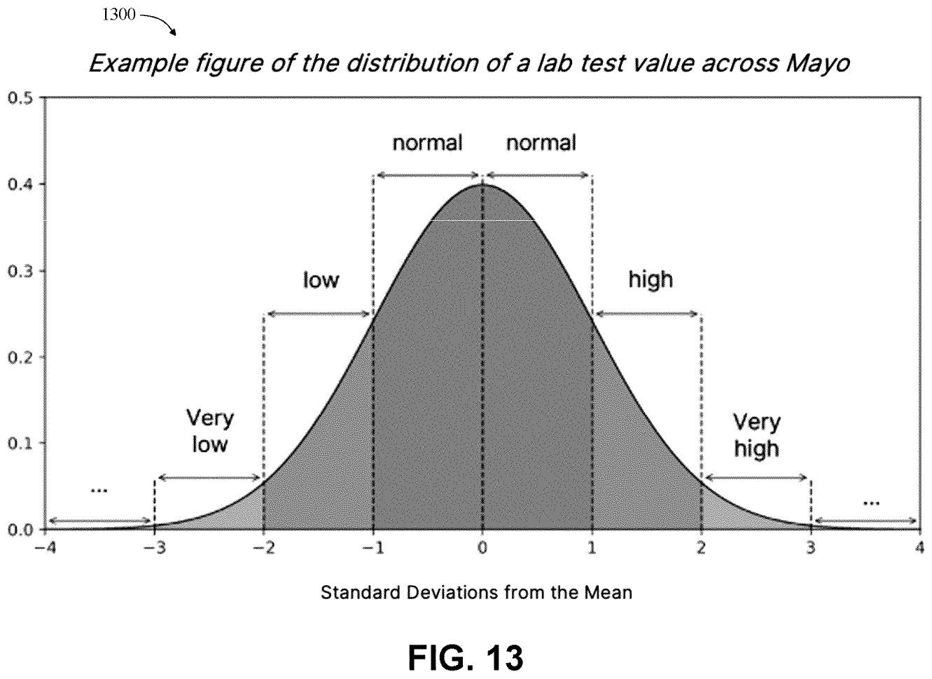

To categorize whether a PH-COPD or COPD patient's lab test was abnormal relative to the matched controls, they may be individually compared to standard distributions generated from the entire Mayo Clinic population. Standard normal distributions may be plotted for each lab test based on the entire Mayo Clinic population using the mean of the lab test values for the 26 different lab tests. If a patient had more than 1 occurrence for the same lab test, the mean of means may be utilized to calculate the population's overall lab test mean. From these unimodal plots, the standard deviations may be calculated. Lab values falling within ±1 STD of the normal distribution's mean may be considered in normal range, whereas ±2 STDs of the normal distribution's mean may categorize the lab value as high or low, respectively, ±3 STDs of the normal distribution's mean may categorize the lab value as very high or very low, respectively, and anything +/−4 STDs away from the mean may be considered ‘other’ (FIG. 13, embodiment 1300).

### 4.1.2 Univariate Analysis and Logistic Regression

Univariate analysis may be an essential first step to understand the data and identify potential covariates that might be predictive or influential in the modeling process. All features and their associated values (means, STD, medians) may be compared in a one-off analysis to distinguish between the PH-COPD and COPD cohorts. Cohen's d, t-tests, chi-squared and odds ratios may be performed depending on the type of feature (continuous versus categorical). A multivariate logistic regression analysis may be used to examine the influencing factors between these two cohorts.

### 4.2 Train a Preliminary PH Detection Algorithm

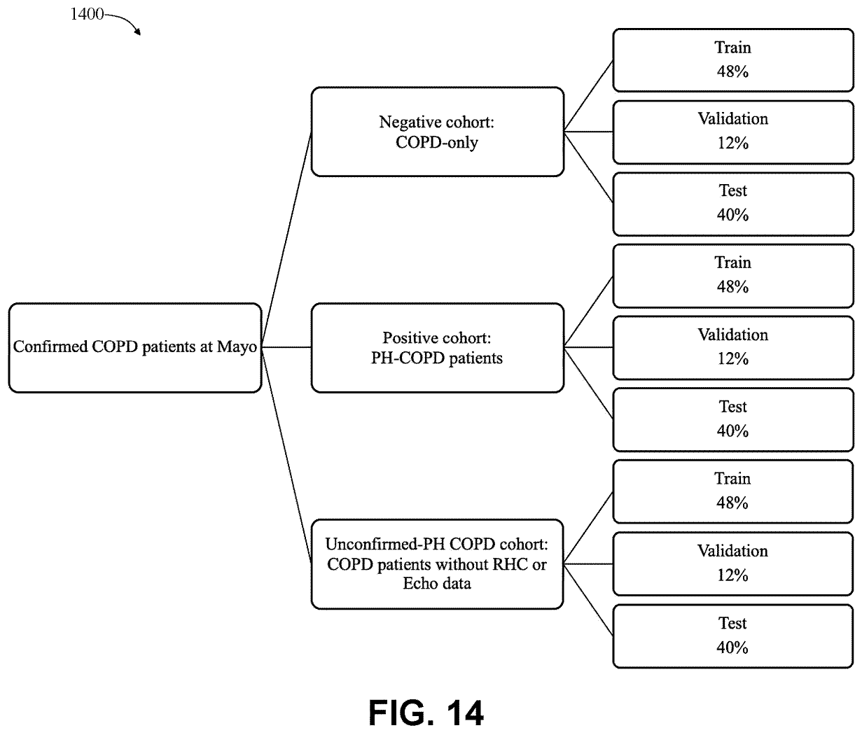

Three types of PH detection algorithms may be trained on feature vectors comprised of the covariates listed in Section 3.6 (taken from EHR/ECG data from date of diagnosis to 3 months afterwards) for three sub-cohorts defined by “PH-COPD”, “COPD-only” and “Screening/unconfirmed PH”: ECG-only, EHR-only and ECG plus EHR. These three cohorts may then be then split into training, validation, and testing groups (FIG. 14, embodiment 1400).

The training/validation/test split is a technique to evaluate the performance of the machine learning model since you cannot evaluate the predictive performance of a model with the same data used for training. Therefore, randomly splitting the data may be a commonly used method for unbiased evaluation at a relative proportion of 48%/12%/40%. The training set may consist of the set of patients and relevant data used for training the model. The validation set may contain the group of patients used to provide an unbiased evaluation of the model fitted on the training dataset while model hyperparameters are tuned. Lastly, the testing dataset may include the unique set of patients used to provide an unbiased evaluation of the final model that was fitted on the training dataset. This may ensure that all three sets are representative of the entire dataset and provides a good way to measure the accuracy of the model.

The feature space may be normalized by patient-level data, such as visit frequency within the time window, i.e. per patient per month. For the EHR-based algorithms, both supervised (hypothesis driven) and contrastive self-supervised (hypothesis-free) approaches may be used.
Logistic regression:
   Pros:
   Interpretability
   Makes no assumptions about distributions of classes in feature space
   Insensitive to missing values
   Cons:
   Cannot accommodate non-numerical values (requires feature scaling or transformation)
   Sensitive to outliers
   Can overfit in high-dimensional datasets

Convolutional neural network (CNN):
Pros:
   CNNs do not require human supervision for the task of identifying important features.
   They are very accurate at image recognition and classification.
   Weight sharing is another major advantage.
   CNNs minimize computation in comparison with a regular neural network.
   They make use of the same knowledge across all image locations.
Cons:
   A lot of training data is needed for the CNN to be effective.
   Tend to be much slower because of operations like maxpool
   Computationally expensive
   Non-expressive learning and logics
   Prone to overfitting because they tend to be deployed on massive features.

Supervised (hypothesis-driven): Uses labeled data with randomized weights to build a function that classifies the output. For example, An ECG for Patient A who has PH COPD versus EHR data for Patient B who is in the COPD only Cohort. An artificial neural network (ANN) is made up of layers of nodes. Each node has a set of weights and a threshold. The input data is transformed by the weights using a mathematical function. If the resulting value exceeds the threshold, the transformed value is passed to the next layer. A feedback loop is employed to improve performance over successive iterations, refining the weights and thresholds. Supervised models require a good amount of training data. The supervised approach incorporates the full data dictionary including lab values, observations, and augmented curation-derived diagnoses.

Contrastive self-supervised (hypothesis-free): Contrastive self-supervised learning takes two sets of labeled inputs. The first step is to train a model to transform those inputs into numerical vectors and minimize the distance between those vectors for inputs with the same label. The resulting vector is called an “embedding”. Once an optimal embedding is determined, it can be used as an input to a supervised model. Additionally, while a supervised network is normally initiated with random weights, this model can be initialized with weights learned during the self-supervised learning and fine-tuned to the new task. This type of model is also benefited by limited training data.

The platform has previously developed a self-supervised embedding for ECG and EHR data using 9M ECGs from 2.4M patients at Mayo Clinic. The EHR vector is the sequence of a patient's ICD codes, medications, and procedures. This “vocabulary” is made up of 28,593 possible features. The embedding clusters patients with similar EHR journeys and ECG signatures closer together. The embedding can be applied separately to ECG and EHR data. Thus, we used this embedding for all 3 model variations. By using this process, it is as if the ECG embeddings contain info about the EHR journey, and vice versa.

For generalizability to health systems beyond the Mayo Clinic, preference may be given to pre-existing, widely available structured features (such as diagnosis codes) and those with high coverage for the patients/windows used (>15%).

In cases where data had missing values, there may be two potential approaches: The associated data type may be inputted as a null for that variable for the given patient. The value may be filled in with the median (for numerical) or mode (categorical) for the training set as a whole.

The approaches used may be determined by a combination of the feature's clinical relevance/importance to PH-COPD and the percentage of missing values for that feature.

Model performance may be assessed on a unique holdout set of patients to determine generalizability.

### 4.2.1 Statistical Measures of ML Model Validation

A true positive (TP) is where the model correctly predicts the patient as having PH. Similarly, a true negative (TN) is an outcome where the model correctly predicts that the patient does not have PH. A false positive (FP) is where the model incorrectly predicts the patient as having PH. And a false negative (FN) is where the model incorrectly assigns a true PH patient as being a control patient.

The recall, also termed the true positive rate (TPR) or sensitivity, is the ratio: TP/(TP+FN). Thus, sensitivity assesses the ability of the classifier to find all the positive patients—a highly sensitive test indicates there are few false negative results, and most disease patients are identified correctly. The specificity of a test: TN/(FP+TN) is its ability to appropriately designate an individual who does not have a disease as negative. The false positive rate (FPR)=FP/(FP+TN) and is a measure of accuracy for the diagnostic model, meaning the probability of falsely rejecting the null hypothesis.

The area under the curve (AUC) may provide an aggregate measure of performance across all possible classification thresholds. One way of interpreting AUC may be to interpret it as the probability that the model ranks a random positive example more highly than a random negative example. 100% prediction accuracy has an AUC of 1.0.

The Diagnostic Odds Ratio (DOR) may be calculated according to the formula: (TP/FN)/(FP/TN). DOR may depend significantly on the sensitivity and specificity of a test. A test with high specificity and sensitivity with low rate of false positives and false negatives may have high a DOR.

Positive predictive value (PPV), also known as precision, refers to the probability that a patient who tests positive for pulmonary hypertension has the condition, i.e. it measures the proportion of true positives among all the patients who tested positive and is calculated as TP/(TP+FP). Negative predictive value (NPV) indicates the probability that a patient who tests negative for pulmonary hypertension is a true control. It measures the proportion of true negatives among all the patients who tested negative and is calculated as: TN/(TN+FN).

Youden's J=TPR-FPR finds the optimal threshold using the best TPR with a low FPR.

### 4.2.2 Attention Mapping

Supervised and self-supervised ensemble transformer EHR models may be limited in their interpretability. Attention mapping may allow for identification of these salient features by using an axiomatic model interpretability algorithm to assign importance scores to each input feature by approximating the integral of gradients of the model's output with respect to the inputs along the path from given baselines or references to inputs.

### 4.3 Estimate Prevalence of PH-COPD Among Patients with COPD Across the Best Performing Models

Prevalence estimation may be based on the results from the three best performing models (ECG-only, EHR+ECG, EHR-only). To estimate the prevalence of PH in a COPD population, we divided the patients identified as PH-COPD in the screening cohort based on each model's output plus the 3012 known PH positive patients, divided by either the number of patients that have a COPD diagnosis between 2015-2019, do not have a PH diagnosis prior to COPD diagnosis, and have an ECG on or after COPD index within the study window (n=53327) or the number of patients that have a COPD diagnosis between 2015-2019 but do not have a PH diagnosis prior to COPD index (n=76,818). These predicted prevalence estimations may then be adjusted using the models' sensitivity (%) to get a resultant sensitivity-corrected prevalence.

### 5. RESULTS

### 5.1 Construction of Study Sample

Of 99,970 adults with COPD between 2015-2019 at Mayo Clinic, 3,012 PH-COPD patients were found with 1) mean pulmonary arterial pressure (mPAP)>20 mmHg on RHC or tricuspid regurgitation velocity (TRV)>3.4 m/s via echocardiogram, 2) COPD diagnosis before or ≤3 months after PH diagnosis, and 3) no Group 1, 2, or 4 PH. A COPD-only cohort of 6,127 patients without PH was identified with mPAP≤20 mmHg on all RHCs and no TRV>3.4 m/s, or if no RHC, all TRV≤2.8 m/s. A screening cohort of 31,362 patients had COPD but with no rule-out or confirmatory RHC or echo (Table 1).

TABLE 1
Patient counts of PH-COPD, COPD-only and screening cohorts

| Cohort | Patient Counts 2015-2019 |
|---|---|
| PH-COPD | 3012 |
| COPD-only | 6127 |
| Screening | 31362 |

### 5.2 Main Results

### 5.2.2 Univariate Analysis and Logistic Regression

### 5.2.2.1 Univariate Analysis

A univariate analysis was performed comparing continuous and categorical features between PH-COPD patients to COPD patients (Tables 3,4). Cohorts were similar with respect to race and sex and only small differences were observed between symptoms. The largest differences were found for laboratory tests: NT-proBNP (PH-COPD vs COPD-only mean: 5520.29 vs 2345.51 pg/mL), creatinine (1.43 vs 1.12 mg/dL), hemoglobin (11.8 vs 12.75 g/dL), erythrocyte distribution width (15.38 vs 14.452%); and Echo/ECG measurements: LV mass (221.13 vs 181.25), LV mass index (112.58 vs 95.23 g/m²), LA volume (85.38 vs 65.96 mL), RVSP (45.99 vs 31.62 mmHg), RA volume (73.65 vs 51.42 mL), RA volume index (37.71 vs 27.53 mL/m²), and QRS duration (109.06 vs 97.55 ms), with all p<0.001. These findings indicate PH-COPD patients have distinct clinical profiles and further understanding of this group will help clinicians identify patients who may benefit from PH-COPD screening.

TABLE 3
Univariate analysis of continuous variables between PH-COPD and COPD-only patients

| | | | | Comparison metrics¹ | | | |
|---|---|---|---|---|---|---|---|
| PH-COPD | | COPD-only | | | | p-value | p-value |
| mean | std | mean | std | cohen's d | t-test | (t-test) | (u-test) |

Demographics

| | | | | | | | | |
|---|---|---|---|---|---|---|---|---|
| Age at COPD diagnosis | 71.7 | 11.11 | 67.12 | 12.52 | 0.3868 | 17.7465 | 0 | 0 |
| Time from first record to | 210.18 | 143.2 | 193.97 | 145.83 | 0.1122 | 5.0561 | 0 | 0 |
| COPD diagnosis (months) | | | | | | | | |
| Time from COPD diagnosis | 96.08 | 65.24 | 75.31 | 53.61 | 0.3478 | 15.1357 | 0 | 0 |
| to last record at Mayo | | | | | | | | |
| (months) | | | | | | | | |

Observations

| | | | | | | | | |
|---|---|---|---|---|---|---|---|---|
| Heart rate (range: | 80.2 | 14.49 | 80.39 | 14.7 | −0.0129 | −0.4643 | 0.6425 | 0.8221 |
| 2.5-97.5 percentile) | | | | | | | | |
| Height (range: 2.5-97.5 | 1.68 | 0.08 | 1.68 | 0.08 | −0.0031 | −0.1191 | 0.9052 | 0.9343 |
| percentile) | | | | | | | | |
| Weight (range: 2.5-97.5 | 84.15 | 23.02 | 82.41 | 21.65 | 0.0779 | 3.1644 | 0.0016 | 0.0063 |
| percentile) | | | | | | | | |
| BMI (range: 5.0-95.0 | 26.4 | 7.5 | 26.22 | 6.97 | 0.0255 | 0.9956 | 0.3195 | 0.0535 |
| percentile) | | | | | | | | |
| Systolic blood pressure | 129.5 | 17.1 | 127.03 | 16.38 | 0.1473 | 3.9795 | 0.0001 | 0.0002 |
| (range: 2.5-97.5 percentile) | | | | | | | | |
| Diastolic blood pressure | 68.73 | 8.89 | 72.55 | 9.05 | −0.4264 | −11.6341 | 0 | 0 |
| (range: 2.5-97.5 percentile) | | | | | | | | |

Healthcare resource utilization

| | | | | | | | | |
|---|---|---|---|---|---|---|---|---|
| Days hospitalized | 4.03 | 7.83 | 1.57 | 4.37 | 0.3875 | 16.0375 | 0 | 0 |
| Emergency department visits | 1.22 | 1.99 | 0.65 | 1.35 | 0.3315 | 14.038 | 0 | 0 |
| Visits to Mayo | 9.39 | 11.84 | 5.27 | 7.83 | 0.4103 | 17.3193 | 0 | 0 |

Other procedures/tests

| | | | | | | | | |
|---|---|---|---|---|---|---|---|---|
| CT scan: lung/chest CT | 0.58 | 0.94 | 0.4 | 0.77 | 0.2073 | 9.0134 | 0 | 0 |
| scan, cardiac CT | | | | | | | | |
| X-ray: lung/chest x-ray | 2.31 | 3.12 | 1.1 | 1.86 | 0.4696 | 19.569 | 0 | 0 |
| Oxygen use | 1.85 | 3.73 | 0.8 | 2.51 | 0.3293 | 13.9334 | 0 | 0 |

Laboratory tests:

| | | | | | | | | |
|---|---|---|---|---|---|---|---|---|
| NT-proBNP- N-terminal pro | 5520.29 | 7699.51 | 2345.51 | 5208.64 | 0.483 | 13.6064 | 0 | 0 |
| b-type natriuretic peptide | | | | | | | | |
| (range: 2.5-97.5 percentile) | | | | | | | | |
| Creatinine (range: 2.5-97.5 | 1.43 | 1.15 | 1.12 | 0.9 | 0.3056 | 12.0559 | 0 | 0 |
| percentile) | | | | | | | | |
| Hemoglobin (range: 2.5-97.5 | 11.8 | 2.13 | 12.75 | 2.02 | −0.4558 | −18.331 | 0 | 0 |
| percentile) | | | | | | | | |
| Hematocrit (range: 2.5-97.5 | 36.58 | 6.22 | 38.63 | 5.75 | −0.3428 | −13.6829 | 0 | 0 |
| percentile) | | | | | | | | |
| Sodium (range: 2.5-97.5 | 139.13 | 3.86 | 139.49 | 3.6 | −0.0951 | −3.7999 | 0.0001 | 0.0001 |
| percentile) | | | | | | | | |
| Red blood cell width | 15.38 | 1.96 | 14.45 | 1.9 | 0.4799 | 19.2563 | 0 | 0 |
| distribution (range: 2.5-97.5 | | | | | | | | |
| percentile) | | | | | | | | |

Medication history²

| | | | | | | | | |
|---|---|---|---|---|---|---|---|---|
| Diuretics [furosemide | 3.6 | 6.61 | 1.03 | 3.09 | 0.4979 | 20.2705 | 0 | 0 |
| (Lasix), bumetanide | | | | | | | | |
| (Bumex), and spironolactone | | | | | | | | |
| (Aldactone)] | | | | | | | | |
| Inhaled Corticosteroids | 2.26 | 5.57 | 1.13 | 3.64 | 0.2409 | 10.1533 | 0 | 0 |
| Bronchodilators | 3.88 | 6.9 | 1.83 | 4.17 | 0.3605 | 15.0481 | 0 | 0 |
| COPD medications | 2.87 | 6.17 | 1.69 | 4.3 | 0.2213 | 9.4026 | 0 | 0 |
| [Phosphodiesterase-5 | | | | | | | | |
| inhibitors, Theophylline, | | | | | | | | |
| and oral steroids] | | | | | | | | |

Echo (as available)³

| | | | | | | | | |
|---|---|---|---|---|---|---|---|---|
| SV- stroke volume (derived, | 73.52 | 21.75 | 69.54 | 21.03 | 0.1858 | 2.8702 | 0.0042 | 0.0022 |
| include method of | | | | | | | | |
| acquisition) | | | | | | | | |
| SV index (stroke volume | 40.04 | 11.09 | 39.55 | 9.87 | 0.0468 | 0.7058 | 0.4805 | 0.7088 |
| index) | | | | | | | | |
| LVEF- left ventricular | 53.7 | 13.16 | 54.4 | 11.96 | −0.0558 | −0.8629 | 0.3884 | 0.5313 |
| ejection fraction | | | | | | | | |
| LV (left ventricular) mass | 221.13 | 78.1 | 181.25 | 66.55 | 0.5496 | 8.673 | 0 | 0 |
| LV (left ventricular) mass | 112.58 | 36.32 | 95.23 | 29.99 | 0.5207 | 8.2411 | 0 | 0 |
| index | | | | | | | | |
| LA (left atrium) volume | 85.38 | 37.09 | 65.96 | 33.69 | 0.5484 | 8.172 | 0 | 0 |
| LA (left atrium) volume | 44.24 | 19.87 | 35.07 | 16.01 | 0.5083 | 7.6386 | 0 | 0 |
| index | | | | | | | | |
| LVEDV- left ventricular | 131.01 | 59.85 | 113.07 | 48.26 | 0.3301 | 4.2267 | 0 | 0.0001 |
| end-diastolic volume lvedv | | | | | | | | |
| RVSP- right ventricular | 45.99 | 8.93 | 31.62 | 6.68 | 1.8217 | 16.6617 | 0 | 0 |
| systolic pressure rvsp | | | | | | | | |
| RV/LV ratio - right | 44.3 | 10.67 | 43.83 | 9.61 | 0.0462 | 0.4162 | 0.6775 | 0.8215 |
| ventricular to left ventricular | | | | | | | | |
| diameter ratio rvlv_ratio | | | | | | | | |
| ra_vol | 73.65 | 43.39 | 51.42 | 29.49 | 0.5993 | 4.0051 | 0.0001 | 0.0002 |
| ra_vol_index | 37.71 | 20.74 | 27.53 | 14.97 | 0.5624 | 3.7199 | 0.0003 | 0.0004 |

ECG (as available):

| | | | | | | | | |
|---|---|---|---|---|---|---|---|---|
| QT interval | 405.78 | 51.05 | 395.88 | 44.86 | 0.2061 | 7.744 | 0 | 0 |
| QRS durations | 109.06 | 31.37 | 97.55 | 24.31 | 0.4102 | 15.1938 | 0 | 0 |

¹values that are 0 are at least <0.00005
²Mean orders per patient
³Echocardiogram measurements used are from the non-diagnostic echocardiogram, if available

TABLE 4
Univariate analysis of categorical variables
between PH-COPD and COPD-only patients

| | | | |
|---|---|---|---|
| odds | p-value | | |
| ratio | (odds | | p-value |
| (OR)¹ | ratio)¹ | chi-sq¹ | (chi-sq)¹ |

Demographics

| | | | | |
|---|---|---|---|---|
| Race (White) | 0.93 | 0.4241 | 0.6 | 0.44 |
| Ethnicity (Not Hispanic or | 1.45 | 0.0005 | 11.35 | 0.0008 |
| Latino) | | | | |
| Gender (Female) | 0.91 | 0.0385 | 4.2 | 0.0405 |

Findings on imaging tests

| | | | | |
|---|---|---|---|---|
| Pulmonary artery enlargement/ | 8.19 | 0 | 28.05 | 0 |
| dilated pulmonary artery | | | | |
| cardiac chamber enlargement | 3.94 | 0 | 88.56 | 0 |
| right ventricular enlargement | 9.53 | 0 | 16.63 | 0 |
| chronic pulmonary embolism | 2.03 | 0.6027 | 0.04 | 0.8467 |
| mosaicism | 3.09 | 0 | 53.05 | 0 |
| centrilobular ground glass | 0 | 1 | 0 | 1 |
| opacities | | | | |
| Interstitial lung disease/ILD | 3.08 | 0 | 39.21 | 0 |
| fibrosis/fibrotic tissue | 3.5 | 0 | 31.94 | 0 |
| honeycombing | 2.17 | 0.0017 | 9.8 | 0.0017 |
| traction bronchiectasis | 2.58 | 0 | 26.37 | 0 |
| combined pulmonary fibrosis | 1.32 | 0 | 19.89 | 0 |
| and emphysema | | | | |
| usual interstitial pneumonia | 3.46 | 0.0004 | 12.89 | 0.0003 |
| (UIP) | | | | |
| hypersensitivity pneumonitis | 1.53 | 0.691 | 0.02 | 0.8766 |
| organizing pneumonia | 2.04 | 0.4521 | 0.42 | 0.5159 |
| interstitial pneumonia | 2.71 | 0.2283 | 0.92 | 0.3373 |
| non specific interstitial | 10.19 | 0.0169 | 4.8 | 0.0284 |
| pneumonia | | | | |
| reticular opacities | 2.24 | 0 | 28.41 | 0 |
| linear opacities | 1.45 | 0.0206 | 5.2 | 0.0226 |

Symptoms (by ICD or NLP)

| | | | | |
|---|---|---|---|---|
| Dyspnea | 1.88 | 0 | 197.03 | 0 |
| Chest pain | 1.06 | 0.3917 | 0.69 | 0.4059 |
| Edema of lower limbs | 2.38 | 0 | 143.49 | 0 |
| Fatigue | 1.14 | 0.0827 | 2.96 | 0.0853 |
| Dizziness | 1.16 | 0.0997 | 2.7 | 0.1004 |
| Fainting/Syncope | 1.14 | 0.1743 | 1.78 | 0.1823 |
| Heart palpitations | 1 | 0.9629 | 0 | 1 |
| Cyanosis | 8.16 | 0.0031 | 8.01 | 0.0047 |

¹values that are 0 are at least <0.00005

### 5.2.2.2 Logistic Regression

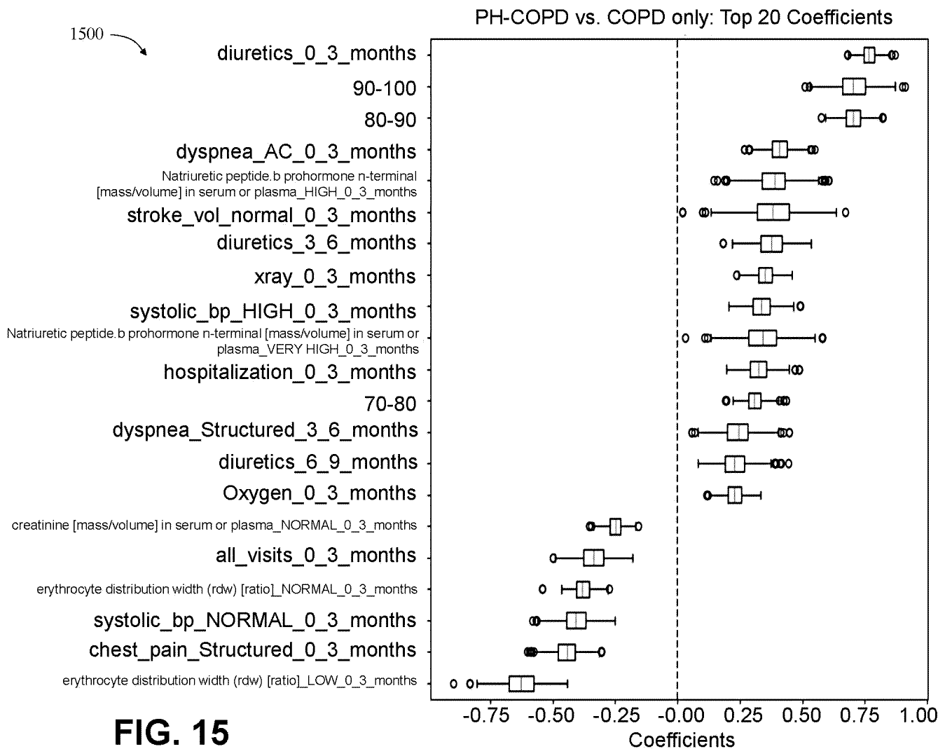

Overall, the multivariate logistic regression showed an AUC of 0.76, suggesting relatively good discrimination ability. FIG. 15, embodiment 1500, highlights the top 20 coefficients identified through multivariate logistic regression analysis on all clinical variables listed in Tables 3 and 4. Coefficients that are greater than 0 predict PH-COPD while those less than 0 predict COPD only. Use of diuretics, comorbid dyspnea, high systolic blood pressure, elevated NT proBNP, oxygen dependence and increased hospitalization were all identified as variables predictive of PH-COPD. There is high concordance between model features and physician decision making. The coefficients marked as ‘NR’ were features that did not reach 50% agreement from panelists.

Worsening lung diffusion capacity from pulmonary function tests were not included for model building due to low coverage within the de-identified data source.

TABLE 5
Features with high physician agreement
and significant model coefficients

| | Physician | |
|---|---|---|
| | Agreement | Model Coefficient |
| | (N = 8) | [95% CI]^{a} |
| High O₂ requirement | 75% | 0.23[0.15, 0.3]* |
| Worsening lung diffusion | 75% | NA^{b} |
| capacity | | |
| High BNP/NT-proBNP | 75% | 0.39[0.24, 0.54] |
| Increased dyspnea on exertion | 50% | 0.41[0.32, 0.49] |
| Lower extremity edema | 50% | 0.14[0.05, 0.23] |
| Signs of right heart dysfunction | 50% | NA^{b} |
| Diuretic use | NR | 0.77[0.69, 0.84] |
| Age (80-89 y) | NR | 0.7[0.61, 0.78] |
| Low erythrocyte distribution width | NR | −0.63[−0.76, −0.5] |
| Low hemoglobin | NR | 0.17[0.08, 0.25] |

NR = Not reported
^{a}All p < 0.05; coefficients >0 predict PH-COPD, <0 COPD only
^{b}Excluded from model: low coverage (<10% of PH-COPD patients)

### 5.2.3 PH Detection Algorithm Development

### 5.2.3.1 EHR Only, ECG Only and ECG+EHR Models

Table 6 shows the performance metrics for the four different models (EHR only [supervised], EHR only [self-supervised], ECG only and ECG+EHR). All AUCs were greater than or equal to 0.79, indicating a high level of discriminative ability between COPD patients with and without PH.

At the optimal threshold (0.295), the ECG-only model showed the highest sensitivity and specificity of 80.2 (95% CI: 76.94-84.39) and 80.02 (95% CI: 77.79-82.08), respectively, demonstrating that 80% of the true negative cases were accurately identified by the model. The diagnostic odds ratio was calculated to quantify the effectiveness of the model as a diagnostic tool. The DOR for this model was 16.44 (95% CI: 13.49-21.11), indicating that the odds of the model correctly identifying PH in COPD patients were approximately 16 times higher than the odds of a false positive. The PPV of the model was 0.61 (95% CI: 0.58-0.64), which reflects that 61% of the patients predicted to have PH by the model have PH. The NPV was 0.91 (95% CI: 0.90-0.93), indicating that 91% of the patients predicted not to have PH by the model were COPD only.

For the EHR only (self-supervised), sensitivity and specificity values (95% CI) were 76.4 (74.53-78.12) and 76.2 (74.99-77.44), respectively. The DOR for this model was 10.4 (95% CI: 9.38-11.76), indicating that the odds of the model correctly identifying PH in COPD patients were approximately 10 times higher than the odds of a false positive. The PPV of the model was 0.63 (95% CI: 0.62-0.65), which reflects that 63% of the patients predicted to have PH by the model have PH. The NPV was 0.86 (95% CI: 0.85-0.87), indicating that 86% of the patients predicted not to have PH by the model were COPD only.

For the ECG+EHR (self-supervised) model, the model's sensitivity was 80.27 (95% CI: 76.58-83.83), meaning it correctly identified roughly 80% of the actual PH cases in COPD patients. The specificity was 80.24 (95% CI: 78.05-82.02), similarly indicating that 80% of the non-PH cases were accurately identified as such by the model. The Diagnostic Odds Ratio was 16.76 (95% CI: 13.35-20.94). This suggests that the odds of the model correctly identifying PH in COPD patients were almost 17 times higher than the odds of a false positive. The PPV of the model was 0.61 (95% CI: 0.58-0.64), which means that 61% of patients predicted to have PH by the model were true positives. The NPV was 0.91 (95% CI: 0.9-0.93), indicating that 90% of patients predicted to be COPD-only were indeed negative cases.

TABLE 6
Performance metrics for the EHR-only, ECG-only and ECG + EHR algorithms

| | EHR-only | | EHR-only (self- | | | | | |
|---|---|---|---|---|---|---|---|---|
| | (supervised) | | supervised) | | ECG-only | | ECG + EHR | |
| Metric | Mean | 95% CI | Mean | 95% CI | Mean | 95% CI | Mean | 95% CI |
| AUC | 0.79 | (0.78-0.81) | 0.84 | (0.83-0.86) | 0.87 | (0.85-0.90) | 0.87 | (0.85-0.90) |
| Sensitivity, % | 72.63 | (70.56-74.53) | 76.85 | (74.53-78.12) | 80.2 | (76.94-84.39) | 80.27 | (76.58-83.83) |
| Specificity, % | 72.2 | (70.77-73.75) | 76.62 | (75.62-77.98) | 80.02 | (77.79-82.08) | 80.24 | (78.05-82.02) |
| Diagnostic | 6.91 | 6.23-7.92 | 10.91 | (9.66-12.20) | 16.44 | (13.49-21.11) | 16.76 | (13.35-20.94) |
| odds ratio | | | | | | | | |
| PPV | 0.58 | 0.57-0.60 | 0.61 | (0.60-0.63) | 0.61 | (0.58-0.64) | 0.61 | (0.58-0.64) |
| NPV | 0.83 | 0.82-0.84 | 0.87 | (0.86-0.88) | 0.91 | (0.90-0.93) | 0.91 | (0.90-0.93) |
| Threshold | 0.214 | | 0.332 | | 0.295 | | 0.29 | |

### 5.2.3.2 Attention Mapping

Attention mapping highlighted the key features that were integral in the EHR only (supervised and self-supervised) models' decision-making process and provided insights into the significant factors associated with PH in COPD patients (Tables 7,8). In the supervised EHR only model, which uses the full data dictionary including unstructured text, lab tests and observations, some of the highest scored features included symptoms such as dyspnea, dizziness, and chest pain. Lab tests like elevated NT pro-BNP, RDW and decreased hematocrit were also highly important variables. Similarly, observations like obesity and elevated diastolic blood pressure came up.

In the self-supervised approach, commonly presenting comorbid conditions were found as salient including ischemic heart disease, CKD, hypertension, atrial fibrillation, and bundle branch blocks which we know are associated with pulmonary hypertension. Several pulmonology related diseases are present as well including pleural effusion and structured diagnosis codes related to breathing difficulties. The use of diuretics is also highly weighted. ECG abnormalities are also present, underscoring the predictive capabilities of the ECG for a disease etiology only diagnosed traditionally by RHC and echo.

TABLE 7
Highest scored features using attention mapping
from the supervised EHR only model

| Description | score |
|---|---|
| diuretics | 200.3756 |
| natriuretic peptide.b prohormone n-terminal [mass/volume] in serum or | 69.742 |
| plasma_OTHER | |
| creatinine [mass/volume] in serum or plasma_OTHER | 44.1736 |
| erythrocyte distribution width (rdw) [ratio]_OTHER | 27.5285 |
| dyspnea_AC | 27.1299 |
| natriuretic peptide.b prohormone n-terminal [mass/volume] in serum or | 16.8456 |
| plasma_HIGH | |
| all_visits | 16.6613 |
| weight_NORMAL | 15.3672 |
| natriuretic peptide.b prohormone n-terminal [mass/volume] in serum or | 14.7214 |
| plasma_VERY HIGH | |
| QRSDURATION_OTHER | 11.6172 |
| copd_meds | 11.0243 |
| Xray | 9.8158 |
| systolic bp_NORMAL | 9.2861 |
| height_NORMAL | 9.2683 |
| hemoglobin in whole blood_NORMAL | 8.029 |
| erythrocyte distribution width (rdw) [ratio]_HIGH | 7.8693 |
| diastolic bp_NORMAL | 7.6605 |
| sodium-serum/plasma-substance concentration-point in time--_NORMAL | 7.2112 |
| erythrocyte distribution width (rdw) [ratio]_VERY HIGH | 7.1204 |
| hematocrit [volume fraction] of blood by automated count_OTHER | 7.0575 |
| hemoglobin in whole blood_VERY LOW | 7.0416 |
| erythrocyte distribution width (rdw) [ratio]_NORMAL | 6.8226 |
| Q_TINTERVAL_OTHER | 6.5147 |
| body mass index (bmi)_NORMAL | 6.1467 |

TABLE 8
Highest scored features using attention mapping
from the self-supervised EHR only model

| Description | score |
|---|---|
| heart failure | 1924.3655 |
| other chronic obstructive pulmonary disease | 1409.8629 |
| atrial fibrillation and flutter | 1345.7885 |
| other pulmonary heart diseases | 1005.8395 |
| complications and ill-defined descriptions of heart disease | 782.8515 |
| atrioventricular and left bundle-branch block | 619.5464 |
| chronic kidney disease (ckd) | 522.976 |
| multiple valve diseases | 491.7289 |
| abnormal results of function studies | 467.7729 |
| electrocardiogram, routine ecg with at least 12 leads; inter | 436.2526 |
| cardiac implantable device nos | 392.5449 |
| chronic ischemic heart disease | 369.466 |
| nonrheumatic mitral valve disorders | 353.1338 |
| cardiac arrest | 344.8259 |
| other interstitial pulmonary diseases | 310.9096 |
| other conduction disorders | 294.7985 |
| cardiomyopathy | 292.8915 |
| rheumatic tricuspid valve diseases | 267.7692 |
| effusion pleural | 264.786 |
| electrocardiogram, routine ecg with at least 12 leads; traci | 262.8833 |
| abnormalities of breathing | 255.3534 |
| ecg routine ecg w/least 12 lds w/i&r | 222.4414 |
| natriuretic peptide | 220.98 |
| Furosemide | 205.8051 |
| essential (primary) hypertension | 205.4059 |

### 5.2.4 Estimate Prevalence of PH-COPD Among Patients with COPD Across a Variety Of Clinical Settings

Based on the three best performing models, the sensitivity-corrected prevalence of PH in COPD populations were as follows: 6.87%, 5.92% and 6.77%, respectively (Table 9). This adjustment indicates a lower prevalence compared to the raw estimates, reflecting the importance of accounting for diagnostic test characteristics, and aligning more with expert consensus.

TABLE 9
Prevalence estimations of PH in COPD populations in the three best performing models

| | | Screening | Percent | Numerator | | | | |
|---|---|---|---|---|---|---|---|---|
| | Screening | Predicted | Positive in | (Probable + | | | | Sensitivity − |
| Best-performing | Predicted | COPD- | Screening | 3012 PH- | | Estimated | | Corrected |
| models | PH-COPD | Only | Cohort | COPD) | Denominator | Prevalence | Sensitivity | Prevalence |
| ECG Only | 810 | 6264 | 11.45% | 3822 | 53,327 | 7.17% | 0.802 | 6.87% |
| EHR only (Self- | 2003 | 28963 | 6.47% | 5015 | 76818 | 6.53% | 0.7685 | 5.92% |
| supervised) | | | | | | | | |
| ECG + EHR | 745 | 6329 | 10.53% | 3757 | 53327 | 7.05% | 0.8027 | 6.77% |

### 6. DISCUSSION

The current study focused on the development of machine learning algorithms for detecting PH in patients with COPD. Features may originate directly from clinicians and their experience in treating the condition of interest. The objective was to identify key features that we could incorporate into the feature space for algorithm development. Overall, the physicians reported 6 clinical features with greater than or equal to 50% agreement, which were increased dyspnea on exertion, elevated NT pro-BNP, edema, signs of right heart failure and a high O₂ requirement.

The univariate analysis and multivariate logistic regression were both preliminary ways of providing an initial understanding of each feature's potential to discriminate between the COPD only and PH COPD populations.

Using a comprehensive feature space comprising demographics, lab tests, observations, diagnoses, echo parameters and discrete ECG values, EHR only, ECG only and ECG+EHR neural networks were trained. These models have shown promising results, as evidenced by the high AUC values, balanced sensitivities and specificities, and strong positive and negative predictive values observed in the top-performing hypothesis-free EHR only, ECG only and ECG+EHR models. These metrics highlight the reliability and potential clinical utility of the models in early detection and management of PH within this patient population.

The integration of attention mapping for feature identification has demonstrated the robustness and reliability of the self-supervised EHR only algorithm. This approach may not only enhance the interpretability of the model but also provide valuable insights for clinical practice, potentially guiding targeted interventions and management strategies for at-risk patients.

The sensitivity-adjusted prevalence estimations, ranging from 5.9% to 6.87%, may provide a more accurate representation of the true burden of PH in COPD patients. This information may be crucial for healthcare planning and resource allocation, ensuring that appropriate measures are taken to address this significant health issue.

As a method of validating the ‘probable-PH’ labels generated from the models run on the screening cohort, prospective EHR data from 2020 through 2023 was used. The presence of mPAP≥25 from RHC or TRV>3.4 m/s from echocardiograms may serve as confirmatory indicators of PH diagnosis for a given patient. From the EHR only model, 161 out of 2003 patients had an available RHC or echo between 2020-2023. Out of those patients, 18.6% were correctly identified as positive PH. Similarly, for the ECG only algorithm, 56 out of 810 patients had an available RHC or echo between 2020-2023. Out of those patients, 26.8% of probable-PH labeled patients had positive results. Lastly, for the ECG+EHR model, 48 out of the 745 patients had an available RHC or echo in the future at Mayo and 27.1% of those were found to be positive. However, it is important to note that the evaluation of model accuracy, which may rely on confirmatory diagnosis via RHC or echocardiogram, may introduce a bias. This bias may arise from the fact that true negative cases, who may not require cardiac evaluation, are not screened, and hence are not included in the population, leading to a decreased PPR for PH-negative cases. Additionally, the model was trained on a preceding feature space window of 12 months to identify undiagnosed PH patients. Looking prospectively to understand model performance may frame the model objective in a predictive lens by potentially using features up to 8 years prior to PH diagnosis. Ultimately, these patients could be used as a hold-out test set for independent testing during a follow-up study.

In summary, these findings indicate that the models developed in this study may be reliable and potentially valuable tools for the detection of PH in COPD patients. The robustness of these models, combined with their clinical interpretability and relevance, underscores their potential impact on improving patient outcomes through timely and targeted interventions. Future work should be conducted to optimize model performance including incorporation of pulmonary function tests into the feature space.

### 6.1 Biases or Limitations

### 6.1.1 Methods to Minimize Bias

There was the potential for information bias, as data from the Mayo Clinic are generated from real-world clinical settings and are subject to miscoding and errors. Further, it is possible that there were missing data and other data quality and assessment issues pertaining to signs, symptoms, diagnoses, and procedures. Methods to address missingness were discussed in Section 4.2. However, it is unlikely that any of these errors are systemic and did not likely impact the findings.

### 6.1.2 Limitations

We anticipated the following limitations of the study based on the design proposed:
Patient's treatment and medical history are limited to data available in the Mayo Clinic EHR. While any medication administered at Mayo should be captured, it may not be possible to establish the full patient history prior to COPD diagnosis.
Patient outcomes and disease progression may be incomplete for patients who are lost to follow-up since they may continue treatment outside of the Mayo clinic.
Data will be exclusively from a single, albeit multi-center, academic health system and may not be representative or translatable to community practices or smaller academic centers.
Data longitudinally may vary in both extent and frequency due to the variability in timing for real-world patient care (in contrast with controlled trials with set time points for data collection)
ML Model limitations:
   Logistic regression
      Cannot accommodate non-numerical values (requires feature scaling or transformation)
      Sensitive to outliers.
      Can overfit in high-dimensional datasets
   Convolutional neural network
      A lot of training data is needed for the CNN to be effective.
      Computationally expensive
      Non-expressive learning and logics
      Prone to overfitting because they tend to be deployed on massive feature spaces

### 7. CONCLUSION

These findings indicate that PH-COPD patients have distinct clinical profiles from COPD-only patients and further understanding of this group may help clinicians identify patients who may benefit from PH-COPD screening. The results also show that models can be leveraged in a clinical system to improve detection of PH among COPD patients. This could facilitate PH-COPD patient referrals to PH specialty centers for individualized care in accordance with current guidelines, potentially improving clinical outcomes.

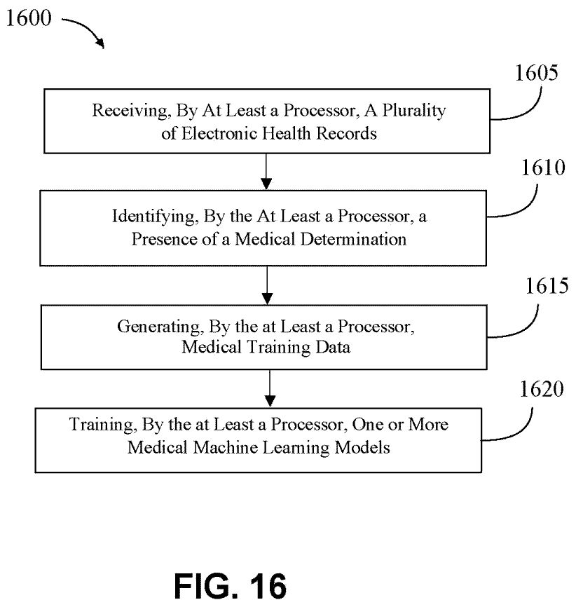

Referring now to FIG. 16, a method 1600 for prediction of medical diseases is described. At step 1605 method 1600 includes receiving, by at least a processor, a plurality of electronic health records associated with a plurality of patients from a patient database. This may be implemented with reference to FIGS. 1-16 and without limitation.

With continued reference to FIG. 16, at step 1610 method 1600 includes identifying, by the at least a processor, a presence of a medical diagnosis for each electronic health record of the plurality of electronic health records, wherein determining the diagnosis includes identifying one or more medical factors within each heath record and assigning the medical diagnosis to each electronic health record as a function of the one or more medical factors. This may be implemented with reference to FIGS. 1-16 and without limitation.

With continued reference to FIG. 16, at step 1615 method 1600 includes generating, by the at least a processor, medical training data having a plurality of electronic health records correlated to a plurality of medical determination wherein at least a portion of the plurality of electronic health records lack a medical determination. In one or more embodiments, generating, by the at least a processor, the medical training data further includes identifying a medical history timeframe associated with each electronic health record of the plurality of electronic health record and segmenting each electronic health record of the plurality of electronic health records as a function of the medical history timeframe and an observation time. In one or more embodiments, the observation time includes a time frame covering at least one year prior to at least one medical factor of the one or more medical factors. This may be implemented with reference to FIGS. 1-16 and without limitation.

With continued reference to FIG. 16, at step 1620 method 1600 includes training, by the at least a processor, one or more medical machine learning models as a function of the medical training data, wherein the one or more medical machine learning models are configured to receive an electronic health record associated with a patient and output a probability of a medical determination. In one or more embodiments one or more medical machine learning models include a transformer-based machine learning model. In one or more embodiments, the transformer-based machine learning model is configured to use attention mechanisms to capture temporal interdependencies within the plurality of electronic health records. In one or more embodiments, capturing temporal interdependencies within the plurality of electronic health records includes generating an attention score of at least one data element within at least one electronic health record of the plurality of electronic health records. In one or more embodiments, the probability of the medical determination includes a softmax score ranging from 0 to 1. In one or more embodiments, the plurality of electronic health records include one or more temporal features and training, by the at least a processor, the one or more medical machine learning models as a function of the medical training data includes training the one or more medical machine learning models as a function of the one or more temporal features. In one or more embodiments, training the one or more medical machine learning models as a function of the one or more temporal features includes assigning a weight to each temporal feature of the one or more temporal features. In one or more embodiments, training, by the at least a processor, an ensemble model as a function of the one or more medical machine learning models wherein training the ensemble machine learning model includes receiving learned features from each of the one or more medical machine learning models and training the ensemble machine learning model as a function of the learned features. This may be implemented with reference to FIGS. 1-16 and without limitation.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

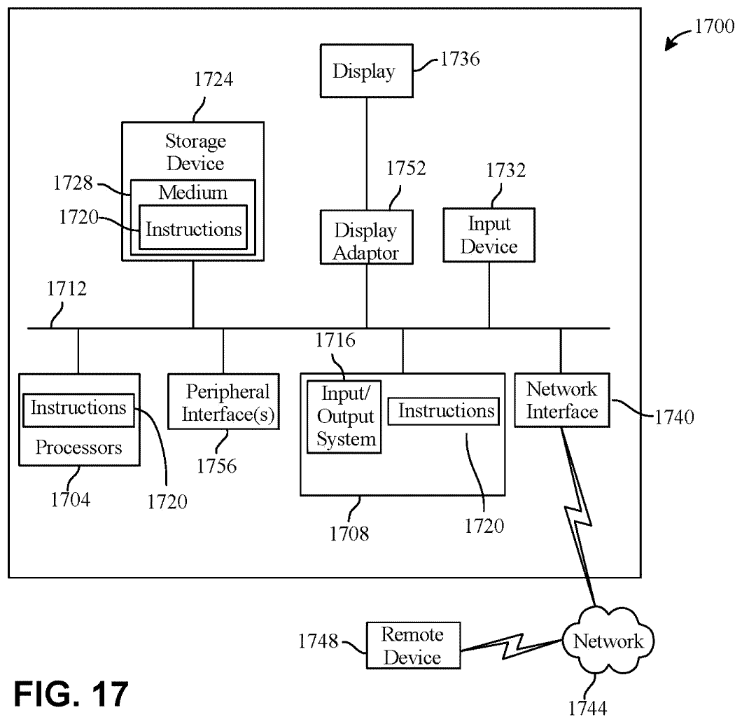

FIG. 17 shows a diagrammatic representation of one embodiment of a computing device in the exemplary form of a computer system 1700 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1700 includes a processor 1704 and a memory 1708 that communicate with each other, and with other components, via a bus 1712. Bus 1712 may include any of several types of bus structures including, but not limited to, a memory bus, a memory controller, a peripheral bus, a local bus, and any combinations thereof, using any of a variety of bus architectures.

Processor 1704 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1704 may be organized according to Von Neumann and/or Harvard architecture as a non-limiting example. Processor 1704 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 1708 may include various components (e.g., machine-readable media) including, but not limited to, a random-access memory component, a read only component, and any combinations thereof. In one example, a basic input/output system 1716 (BIOS), including basic routines that help to transfer information between elements within computer system 1700, such as during start-up, may be stored in memory 1708. Memory 1708 may also include (e.g., stored on one or more machine-readable media) instructions (e.g., software) 1720 embodying any one or more of the aspects and/or methodologies of the present disclosure. In another example, memory 1708 may further include any number of program modules including, but not limited to, an operating system, one or more application programs, other program modules, program data, and any combinations thereof.

Computer system 1700 may also include a storage device 1724. Examples of a storage device (e.g., storage device 1724) include, but are not limited to, a hard disk drive, a magnetic disk drive, an optical disc drive in combination with an optical medium, a solid-state memory device, and any combinations thereof. Storage device 1724 may be connected to bus 1712 by an appropriate interface (not shown). Example interfaces include, but are not limited to, SCSI, advanced technology attachment (ATA), serial ATA, universal serial bus (USB), IEEE 17174 (FIREWIRE), and any combinations thereof. In one example, storage device 1724 (or one or more components thereof) may be removably interfaced with computer system 1700 (e.g., via an external port connector (not shown)). Particularly, storage device 1724 and an associated machine-readable medium 1728 may provide nonvolatile and/or volatile storage of machine-readable instructions, data structures, program modules, and/or other data for computer system 1700. In one example, software 1720 may reside, completely or partially, within machine-readable medium 1728. In another example, software 1720 may reside, completely or partially, within processor 1704.

Computer system 1700 may also include an input device 1732. In one example, a user of computer system 1700 may enter commands and/or other information into computer system 1700 via input device 1732. Examples of an input device 1732 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1732 may be interfaced to bus 1712 via any of a variety of interfaces (not shown) including, but not limited to, a serial interface, a parallel interface, a game port, a USB interface, a FIREWIRE interface, a direct interface to bus 1712, and any combinations thereof. Input device 1732 may include a touch screen interface that may be a part of or separate from display 1736, discussed further below. Input device 1732 may be utilized as a user selection device for selecting one or more graphical representations in a graphical interface as described above.

A user may also input commands and/or other information to computer system 1700 via storage device 1724 (e.g., a removable disk drive, a flash drive, etc.) and/or network interface device 1740. A network interface device, such as network interface device 1740, may be utilized for connecting computer system 1700 to one or more of a variety of networks, such as network 1744, and one or more remote devices 1748 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1744, may employ a wired and/or a wireless mode of communication. In general, any network topology may be used. Information (e.g., data, software 1720, etc.) may be communicated to and/or from computer system 1700 via network interface device 1740.

Computer system 1700 may further include a video display adapter 1752 for communicating a displayable image to a display device, such as display device 1736. Examples of a display device include, but are not limited to, a liquid crystal display (LCD), a cathode ray tube (CRT), a plasma display, a light emitting diode (LED) display, and any combinations thereof. Display adapter 1752 and display device 1736 may be utilized in combination with processor 1704 to provide graphical representations of aspects of the present disclosure. In addition to a display device, computer system 1700 may include one or more other peripheral output devices including, but not limited to, an audio speaker, a printer, and any combinations thereof. Such peripheral output devices may be connected to bus 1712 via a peripheral interface 1756. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A system for prediction of medical diseases, the system comprising:
at least a processor; and
a memory communicatively connected to the at least a processor, the memory containing instructions configuring the at least a processor to:
receive a plurality of electronic health records associated with a plurality of patients from a patient database;
identify a presence of a medical determination for each electronic health record of the plurality of electronic health records, wherein determining the medical determination comprises:
identifying one or more medical factors within each electronic heath record; and
assigning the medical determination to each electronic health record as a function of the one or more medical factors;
segment each electronic health record of the plurality of electronic health records as a function of the medical determination into a gastrointestinal cohort and a control cohort;
generate medical training data as a function of the plurality of electronic health records and the presence of the medical determination, wherein the medical training data comprises a plurality of electronic health records correlated to a plurality of medical determinations, and wherein at least a portion of the plurality of electronic health records within the medical training data lacks a corresponding medical determination; and
train one or more medical machine learning models as a function of the medical training data, wherein the one or more medical machine learning models are configured to receive an electronic health record associated with a patient as an input and output a probability of medical determination, wherein the one or more models comprises:
a first medical machine learning model configured to predict a disease precursor to gastrointestinal related cancer;
a second medical machine learning model configured to predict the cancer, wherein the first medical machine learning model and the second medical machine learning model are trained using differing ratios between the electronic health records from gastrointestinal cohorts and control cohorts of the medical training data; and
an ensemble model trained based on the first medical machine learning model and the second medical machine learning model, wherein the training comprises:
receiving learned features from each of the first medical machine learning model and the second medical machine learning model; and
training the ensemble model as a function of the learned features, wherein the ensemble model is configured to receive probabilities of medical determinations from the first medical machine learning model and the second medical machine learning model as an input and output a weighted probability of medical determination.

2. The system of claim 1, wherein generating the medical training data further comprises:
identifying a medical history timeframe associated with each electronic health record of the plurality of electronic health records; and
segmenting each electronic health record of the plurality of electronic health records as a function of the medical history timeframe and an observation time.

3. The system of claim 2, wherein the observation time comprises a time frame covering at least one month prior to at least one medical factor of the one or more medical factors.

4. The system of claim 1, wherein the one or more medical machine learning models comprise a transformer-based machine learning model.

5. The system of claim 4, wherein the transformer-based machine learning model is configured to capture temporal interdependencies within the plurality of electronic health records, using attention mechanisms.

6. The system of claim 5, wherein capturing temporal interdependencies within the plurality of electronic health records comprises generating an attention score of at least one data element within at least one electronic health record of the plurality of electronic health records.

7. The system of claim 1, wherein the probability of medical determination comprises a softmax score ranging from 0 to 1.

8. The system of claim 1, wherein:
the plurality of electronic health records comprise one or more temporal features; and
training the one or more medical machine learning models as a function of the medical training data comprises training the one or more medical machine learning models as a function of the one or more temporal features.

9. The system of claim 8, wherein training the one or more medical machine learning models as a function of the one or more temporal features comprises assigning a weight to each temporal feature of the one or more temporal features.

10. (canceled)

11. A method for prediction of medical diseases, the method comprising:
receiving, by at least a processor, a plurality of electronic health records associated with a plurality of patients from a patient database;
identifying, by the at least a processor, a presence of a medical determination for each electronic health record of the plurality of electronic health records, wherein determining the medical determination comprises:
identifying one or more medical factors within each electronic heath record; and
assigning the medical determination to each electronic health record as a function of the one or more medical factors;
segmenting, by the at least a processor, each electronic health record of the plurality of electronic health records as a function of the medical determination into a gastrointestinal cohort and a control cohort;
generating, by the at least a processor, medical training data as a function of the plurality of electronic health records and the presence of the medical determination, wherein the medical training data comprises a plurality of electronic health records correlated to a plurality of medical determinations, and wherein at least a portion of the plurality of electronic health records within the medical training data lack a corresponding medical determination; and
training, by the at least a processor, one or more medical machine learning models as a function of the medical training data, wherein the one or more medical machine learning models are configured to receive an electronic health record associated with a patient as an input and output a probability of medical determination, wherein the one or more models comprises:
a first medical machine learning model configured to predict a disease precursor to gastrointestinal related cancer;
a second medical machine learning model configured to predict the cancer, wherein the first medical machine learning model and the second medical machine learning model are trained using differing ratios between the electronic health records from gastrointestinal cohorts and control cohorts of the medical training data; and
an ensemble model trained based on the first medical machine learning model and the second medical machine learning model, wherein the training comprises:
receiving learned features from each of the first medical machine learning model and the second medical machine learning model; and
training the ensemble model as a function of the learned features, wherein the ensemble model is configured to receive probabilities of medical determinations from the first medical machine learning model and the second medical machine learning model as an input and output a weighted probability of medical determination.

12. The method of claim 11, wherein generating, by the at least a processor, the medical training data further comprises:
identifying a medical history timeframe associated with each electronic health record of the plurality of electronic health records; and
segmenting each electronic health record of the plurality of electronic health records as a function of the medical history timeframe and an observation time.

13. The method of claim 12, wherein the observation time comprises a time frame covering at least one month prior to at least one medical factor of the one or more medical factors.

14. The method of claim 11, wherein the one or more medical machine learning models comprise a transformer-based machine learning model.

15. The method of claim 14, wherein the transformer-based machine learning model is configured to capture temporal interdependencies within the plurality of electronic health records using attention mechanisms.

16. The method of claim 15, wherein capturing temporal interdependencies within the plurality of electronic health records comprises generating an attention score of at least one data element within at least one electronic health record of the plurality of electronic health records.

17. The method of claim 11, wherein the probability of medical determination comprises a softmax score ranging from 0 to 1.

18. The method of claim 11, wherein:
the plurality of electronic health records comprise one or more temporal features; and
training, by the at least a processor, the one or more medical machine learning models as a function of the medical training data comprises training the one or more medical machine learning models as a function of the one or more temporal features.

19. The method of claim 18, wherein training the one or more medical machine learning models as a function of the one or more temporal features comprises assigning a weight to each temporal feature of the one or more temporal features.

20. (canceled)
